(19) United States
(12) Patent Application Publication
(10) Pub. No.: US 2026/0219251 A1
(43) Pub. Date: Jul. 30, 2026

(54) METHODS OF ESTIMATING SHELF-LIFE OF PACKAGED FOODS AND BEVERAGES

(71) Applicant: PURDUE RESEARCH FOUNDATION, West Lafayette, IN (US)

(72) Inventors: Dharmendra Kumar Mishra, West Lafayette, IN (US); Ferhan Ozadali, Newburgh, IN (US); Patnarin Benyathiar, West Lafayette, IN (US); Anbuhkani Muniandy, Plainfield, IN (US)

(21) Appl. No.: 19/148,631

(22) PCT Filed: Jan. 16, 2024
(86) PCT No.: PCT/US2024/011609

§ 371 Date: Jul. 16, 2025

Related U.S. Application Data
(60) Provisional application No. 63/480,052, filed on Jan. 16, 2023.

(51) Int. Cl. G01N 33/02

(52) U.S. Cl. CPC G01N 33/02

(57) ABSTRACT

Methods of estimating shelf-life of packaged food products in a sealed polymeric package. Such a method includes maintaining the sealed polymeric package in an oxygen atmosphere at a pressure greater than surrounding ambient atmospheric pressure during a test period of time. The sealed polymeric package is maintained at a temperature above room temperature during the test period. A level of degradation of the packaged food product inside the sealed polymeric package is measured at the end of the test period. An estimated shelf-life of the packaged food product is extrapolated as a function of the amount of degradation measured, the temperature, and the pressure.

### CROSS-REFERENCE TO RELATED APPLICATIONS

This application claims the benefit of U.S. Provisional Application No. 63/480,052 filed Jan. 16, 2023, the contents of which are incorporated herein by reference.

### BACKGROUND OF THE INVENTION

The invention generally relates to methods of estimating shelf-life of a packaged food product.

Shelf-life of a packaged food product (such as a food or a beverage) indicates a period, under defined storage conditions, during which the product is suitable for consumption while retaining desired sensory, chemical, physical and biological characteristics. The quality of the food should not deteriorate in any way that the consumer would find it unacceptable while still being compliant with any label declarations. The shelf-life of a food product is dependent on both intrinsic and extrinsic factors as they influence the stability of food products over time. Intrinsic factors include pH, water activity, enzymes, microorganisms, and the presence and concentration of reactive compounds within the food product itself. Extrinsic factors include temperature, humidity, light and external partial pressure of oxygen from outside of the package. Temperature is commonly used as an accelerant as a means to accelerate degradation reactions for shelf-life analysis.

Typically, the shelf-life of food is determined by conducting experiments under intended storage conditions and/or under accelerated storage conditions. The former is conducted by measuring the shelf life under storage conditions naturally experienced by the product and is suitable for products with shorter shelf-life, such as perishable foods. The shelf-life determination of foods with long-term stability is often subjected to accelerated shelf-life testing (ASLT) under environmental conditions that are intended to speed up the degradation reactions. Temperature-based mathematical models, such Q₁₀ and Arrhenius, are used to extrapolate the stability of the product under ambient conditions based on results obtained from ASLT. For both tests, the quality indicators of interest are identified and the acceptability limit of deterioration of the quality indicator are defined.

ASLT has been the common method used in the food industry to fast-track the degradation reactions in shelf-stable food products to shorten the overall analysis time. The common acceleration factor used in ASLT is the temperature, where the products are stored at slightly higher temperatures (up to 45° C.) to increase the rate of degradation reactions. During ASLT, the pre-determined quality indicators are monitored to observe the period in which the quality indicators fall below the acceptability limits. The accelerating factor is then established by correlating temperature with the shelf life of foods. It is not recommended to perform ASLT above 40-45° C., since some temperature-induced reactions may not naturally occur in the food product at ambient conditions.

Though the ASLT has been utilized by the food industry for more than forty years, this method can be considered slow, especially for shelf-stable products that have long shelf-life in the range of 12-24 months. Depending on the food manufacturer and various factors, the timeline between the product development conceptualization and commercialization is sometimes approximately 6-12 months. Typically, the temperature-based acceleration factor for most shelf-stable food products is between 2 and 3. If a product has a shelf-life of 18 months, then the ASLT study at 40° C. should be conducted for at least 2-5 months.

Most of the time, the ASLT study is concluded after the product has been launched to the market due to time constraints in the product development timeline. A faster methodology or technology would greatly help the product developers to assess any stability issues and address them quickly. Hence, there is a need for a more rapid method that can provide developers with faster and better predictions prior to launching the product to the market.

Besides temperature, several other accelerant factors are well known to affect the kinetics of quality degradation reactions in shelf-stable products that are currently being under-utilized. These include pressure, relative gas pressure, relative humidity, oxygen concentration, and light intensity. Accelerants such as light and humidity, are used when appropriate for moisture- and/or light-sensitive products. For products containing oxygen-sensitive ingredients such as vitamins, the temperature is still the main accelerant used in the shelf-life analysis. Oxygen as an accelerant has never been given much attention even though most food products, if not all, are sensitive to oxygen. However, as far as the inventors are aware, the application of oxygen pressure in shelf-life analysis has not been done before. This may be because the literature shows that oxygen pressure has little to no effect on oxidation because the rate-limiting step in oxidation, propagation, is independent of oxygen pressure at levels close to atmospheric and higher. So, exposing the sample to oxygen levels above 0.21 kPa does not change the degradation rate of the vitamins. Moreover, the effect of oxygen concentration on the stability of food becomes negligible at high temperatures since the solubility of oxygen decreases with increasing temperature. However, at lower partial pressures (<0.21 kPa) and at moderate temperatures (<50° C.), oxygen becomes the dominant factor in driving oxidation compared to temperature. Though evidence of oxygen-dependent degradation is seen in ground coffee, infant formula, strawberry juice, sweet potato puree, and garlic mashed potatoes, there is no methodology or technology available that combines oxygen pressure and temperature for shelf-life analysis of packaged foods. Lack of information on the methodology that incorporates multiple accelerants into shelf-life testing of packaged food as well as lack of readily available kinetic or predictive mathematical models to extrapolate the shelf-life at ambient conditions based on test data has been identified as the two main reasons why oxygen pressure, as an accelerant, is being under-utilized.

Since further increase in the environmental temperature of ASLT is not recommended as it can lead to an unrealistic aging process by inducing unnecessary thermal degradation, there is a need to develop an alternative rapid approach that can further decrease the overall shelf-life analysis time and ensure it works in favor of the product development timeline. It is also desirable to have a holistic method that induces a realistic aging process, has a model for shelf-life prediction under ambient conditions, and is easy to adopt.

### BRIEF SUMMARY OF THE INVENTION

The intent of this section of the specification is to briefly indicate the nature and substance of the invention, as opposed to an exhaustive statement of all subject matter and aspects of the invention. Therefore, while this section identifies subject matter recited in the claims, additional subject matter and aspects relating to the invention are set forth in other sections of the specification, particularly the detailed description, as well as any drawings.

The present invention provides, but is not limited to, methods capable of estimating shelf-lives of packaged food products.

According to a nonlimiting aspect of the invention, a method of estimating shelf-life of a packaged food product in a sealed polymeric package is provided. The method includes maintaining the sealed polymeric package in an oxygen atmosphere at a pressure greater than surrounding ambient atmospheric pressure during a test period of time. The sealed polymeric package is maintained at a temperature above room temperature during the test period. A level of degradation of the packaged food product inside the sealed polymeric package is measured at the end of the test period. An estimated shelf-life of the packaged food product is extrapolated as a function of the amount of degradation measured, the temperature, and the pressure.

Technical aspects of methods having features as described above preferably include the ability to estimate shelf-life of a packaged food product in a sealed polymeric package with the use of oxygen pressure and temperature to reduce the shelf-life analysis time by 50% for oxygen-sensitive shelf-stable products in polymeric packages. Methods according to aspects of the present invention may also provide effective acceleration of degradation of foods and beverages with packaging to provide faster shelf-life estimation than currently used methods, may be useful in selection of packaging materials for optimal barrier properties, and/or may be used to address food stability issues before product reaches the market.

These and other aspects, arrangements, features, and/or technical effects will become apparent upon detailed inspection of the figures and the following description.

### BRIEF DESCRIPTION OF THE DRAWINGS

FIG. 1 is a graph showing the scaled sensitivity coefficient (SSC) of oxygen diffusion coefficient.

FIGS. 2A through 2F are graphs illustrating the increase in the headspace (1) and dissolved (2) oxygen levels in PET (A), HDPE (B), PP (C) bottles containing water kept at ultra-accelerated shelf-life test (UASLT) conditions of 10 psig (•), 15 psig (*), 20 psig (p), and 30 psig (−) and accelerated shelf-life test (ASLT) conditions (+), in which each data point represents the mean±standard deviation of triplicate measurement.

FIGS. 3A through 3F illustrate sequential estimation of diffusion coefficient (left), experimental vs predicted oxygen concentration over time (center) and corresponding residual plot of experimental (O_{2 (exp)}) and predicted (O_{2 (pred)}) (right) for (A) headspace oxygen and (B) dissolved oxygen in PET bottles UASLT 20 psig. Legends: (.-.) diffusion coefficient, (*) O_{2 (exp)}, (−) O_{2 (pred)}, and (⋄) residuals.

FIGS. 4A through 4F illustrate sequential estimation of diffusion coefficient (left), experimental vs predicted oxygen concentration over time (center) and corresponding residual plot of experimental (O_{2 (exp)}) and predicted (O_{2 (pred)}) (right) for (A) headspace oxygen and (B) dissolved oxygen in HDPE bottles UASLT 20 psig. Legends: (.-.) diffusion coefficient, (*) O_{2 (exp)}, (−) O_{2 (pred)}, and (⋄) residuals.

FIGS. 5A through 5F illustrate sequential estimation of diffusion coefficient (left), experimental vs predicted oxygen concentration over time (center) and corresponding residual plot of experimental (O_{2 (exp)}) and predicted (O_{2 (pred)}) (right) for (A) headspace oxygen and (B) dissolved oxygen in PP bottles UASLT 30 psig. Legends: (.-.) diffusion coefficient, (*) O_{2 (exp)}, (−) O_{2 (pred)}, and (⋄) residuals.

FIG. 6 illustrates the mechanistic model of oxygen diffusion and simultaneous degradation of vitamins in model foods kept at the UASLT condition in accordance with aspects of the invention.

FIGS. 7A through 7D illustrate degradation of nutrients in model food kept at UAST (O), ASLT (Δ), and control conditions (o) for A) Vitamin A, B) Vitamin D3, C) Vitamin B1, D) Vitamin C.

FIGS. 8A and 8B illustrate changes in color in the model food kept at UAST (O), ASLT (Δ), and control conditions (o), represented with A) ΔE and B) normalized L* value.

FIG. 9 is an image that illustrates browning of model food kept at the control (left), ASLT (middle), and UASLT (right) conditions for 50 days.

FIGS. 10A through 10F illustrate changes in vitamins C (⋄), D3 (é) and A (w) in model food kept at UASLT conditions, 1) modeled with mechanistic model, and 2) corresponding residuals.

FIG. 11 illustrates the Scaled Sensitivity Coefficient (SSC) of the parameter, k, from 1^{st} order reaction kinetic model.

FIGS. 12A through 12H illustrate degradation levels for vitamins and L* in samples kept under the UAST (O), ASLT (Δ), and control conditions (o) 1) response variable with 1^{st} order reaction kinetic (−) and 95% asymptotic confidence interval (-.-) and 2) corresponding residual plot.

FIGS. 13A and 13B illustrate the relationship between oxygen diffusivity values (D) and rate of reaction (k) in temperature-oxygen diffusion models A) modified Arrhenius model B) Modified Van't Hoff-Arrhenius model.

### DETAILED DESCRIPTION OF THE INVENTION

The intended purpose of the following detailed description of the invention and the phraseology and terminology employed therein is to describe what is shown in the drawings, which relate to one or more nonlimiting embodiments of the invention, and to describe certain but not all aspects of the embodiment(s) to which the drawings relate. The following detailed description also describes certain investigations relating to the embodiment(s), and identifies certain but not all alternatives of the embodiment(s). As nonlimiting examples, the invention encompasses additional or alternative embodiments in which one or more features or aspects shown and/or described as part of a particular embodiment could be eliminated, and also encompasses additional or alternative embodiments that combine two or more features or aspects described as part of different embodiments. Therefore, the appended claims, and not the detailed description, are intended to particularly point out subject matter regarded to be aspects of the invention, including certain but not necessarily all of the aspects and alternatives described in the detailed description.

A method of estimating shelf-life of a packaged food product in a sealed polymeric package is provided. The sealed polymeric package is maintained in an oxygen atmosphere at a pressure greater than surrounding ambient atmospheric pressure during a test period of time. The sealed polymeric package is also simultaneously maintained at a temperature above room temperature during the test period. A level of degradation of the packaged food product inside the sealed polymeric package is measured at the end of the test period. An estimated shelf-life of the packaged food product is extrapolated as a function of the amount of degradation measured, the temperature, and the pressure. The pressure may be at a known constant pressure. In one example, the pressure is maintained at approximately 20 psig. The temperature may be maintained at a known constant temperature. In some examples, the known constant temperature is between about 20° C. and about 45° C., between about 25° C. and about 40° C., or at about 40° C. The test period may be a preselected period of time, or the test period may vary and depend upon the packaged food product reaching some preselected level of degradation. The degradation may include, for example, any one or more of change of color of the packaged food product and a change of a nutrient, such as one or more vitamins, such as vitamins A, B1, and/or C, of the packaged food product. The step of extrapolating may include applying a temperature-oxygen diffusion model, such as a modified Arrhenius model and/or a modified Van't Hoff Arrhenius model, that is a function of both the temperature and the pressure. It is understood that the term food product can include any one or more of various types of foods or beverages.

Oxygen pressure as an accelerant in an accelerated shelf-life test (ASLT) has never been given much attention even though it has been shown to affect the shelf-life of food products negatively. However, as disclosed herein relative to aspects of the present invention, oxygen pressure in combination with elevated temperatures up to 40-45° C. can be applied to the external environment of a hermetically sealed package to induce a rapid influx of oxygen to the food product to induce a faster aging process. The development of a rapid shelf-life method using a multi-accelerant approach is preferably accompanied by a predictive model for shelf-life extrapolation at a given condition and/or time. A mechanistic model that combines the oxygen transfer phenomena, temperature, and the rate of reactions can be considered.

Since there is a need for the development of a rapid shelf-life analysis method along with a predictive model, and there is a potential to utilize oxygen pressure as an accelerant, the present disclosure focuses on the invention of a rapid method, referred to herein as the Ultra-Accelerated Shelf-Life Test (UASLT), that combines oxygen pressure and temperature as accelerants. The disclosure also focuses on the development of the predictive model(s) that can be paired with the UASLT method for shelf-life prediction which not only strengthens the invention but also increases the adaptability of the technology as routine analysis in the food industry for other packaging materials, food products, storage conditions, etc. The application of high oxygen pressure along with elevated temperature (e.g., 40° C.) increases the amount of oxygen diffusing into packaged food products which can induce rapid degradation of nutrients to further reduce the overall shelf-life analysis time compared to the conventional ASLT method.

Oxygen pressure can be applied to the external environment of a hermetically sealed package to induce a rapid influx of oxygen to the food product to cause a faster aging process. Elevated temperatures up to 40-45° C. can also be used in combination with oxygen pressure as a multi-accelerant approach. When oxygen is incorporated into the shelf-life analysis, then indirect exposure of oxygen to the product via accelerated diffusion can be considered rather than direct exposure to avoid breaking the sterility of the product and causing unnecessary changes to the samples. Studies have reported a decrease in shelf-life with increasing partial pressure of oxygen up to 0.21 kPa. These studies were conducted in a static environment where the partial pressure of the oxygen was maintained throughout the study with direct exposure of oxygen to food. The partial pressure of oxygen inside a sealed food packaging does not remain the same due to the diffusion of oxygen over time. Furthermore, the stability of oxygen-sensitive products with direct exposure to oxygen might be different than indirect exposure. Conducting a study to investigate the effect of the partial pressure of oxygen at a lower level on the stability of food inside the packaging with an influx of oxygen from the environment can be time-consuming but can be accelerated with the use of high external oxygen pressure. The external pressure can increase the diffusion of oxygen through the packaging materials due to a larger pressure differential gradient which directly increases the rate of degradation reactions in food. Incorporating oxygen as a factor may have the potential to be used for rapid shelf analysis of the food product while keeping the temperature slightly elevated. It is typically preferred to keep the exposure of oxygen level to food≤about 21% when incorporating oxygen as an accelerant for shelf-life studies. If the food sample is exposed to oxygen levels above about 21%, it does not cause a significant impact on the degradation rate and can lead to inaccuracies in shelf-life prediction. At the same time, it is preferred that the pressure applied be optimized to ensure that it does not cause mechanical damage to the packaging materials, which could break the sterility of the product during the analysis and can lead to errors.

In a set of experiments leading to the invention, the present disclosure considers the effect of environmental oxygen pressure on oxygen diffusivity in bottles made of polyethylene terephthalate (PET), high density polyethylene (HDPE) and polypropylene (PP) packaging materials. However, the methods of the present invention could be used with other packaging materials and packaging types. The bottles were placed in custom-made high-pressure (10 to 30 psig) chambers with a 100% oxygen environment. Each bottle was filled with water and flushed with nitrogen to reduce the headspace and dissolved oxygen level below 2%. Bottles were subjected to three pressure levels and the increase in the headspace and dissolved oxygen levels up to ~20% from diffusion were monitored using oxygen sensors placed inside the bottles. The accumulated oxygen was used as a response variable to estimate the diffusion coefficient with the inverse problems approach. Modelling of diffusion was performed by numerically solving Fick's diffusion model governing time-dependent diffusion in 2D axisymmetric geometry using the Transport of Diluted Species in COMSOL Multiphysics. The sequential estimation of the diffusion coefficient was performed using matrix inversion lemma based on the Gauss minimization method with an appropriate initial guess. The scaled sensitivity and residual plots were analyzed for the proper estimation of the parameter and verification of the statistical assumptions, respectively. The oxygen diffusion coefficient of PET, HDPE and PP materials that were exposed to the oxygen pressure was in the range of 0.78 to 1.16×10⁻¹³ m²s⁻¹, 0.14 to 0.46×10⁻¹³ m²s⁻¹ and 0.59 to 14.09×10⁻¹⁶ m²s⁻¹, respectively. It was found that the application of oxygen pressure significantly increased the rate of oxygen transfer for all packaging materials compared to the control samples that were not exposed to the pressure. The application of inverse problems enabled the determination of the diffusion coefficient of oxygen with minimal errors and small confidence intervals. The rapid influx of oxygen with the use of pressure and at a constant temperature has the potential to increase the rate of degradation reactions in foods which will significantly reduce the overall analysis time for predictively estimating shelf-life.

In the course of developing the present invention, certain tests were conducted, which are summarized hereinafter.

Polyethylene terephthalate (PET), high density polyethylene (HDPE) and polypropylene (PP) bottles were used for this study. Both the HDPE and PP bottles had an ethylene vinyl alcohol (EVOH) layer. Oxygen sensors were placed in the bottles using silicone adhesives to monitor the headspace and dissolved oxygen levels. The bottles were filled with distilled water and flushed with nitrogen by immersing a tube supplying nitrogen in the water. The bottles were sealed immediately with their corresponding closures. The initial headspace (C_{h,0)}) and dissolved (C_{d,0)}) oxygen levels were controlled below 2%.

The oxygen diffusivity of the bottles was studied under 3 different pressure levels at an elevated temperature of 40° C. under the ultra-accelerated shelf-life test (UASLT) condition as listed in Table 1, below. Control samples were kept at 40° C. under ASLT conditions. The temperature selected for this study is commonly used to determine the shelf-life of food products under accelerated conditions. Custom-made pressure chambers were used to create the environment needed for the UASLT study by supplying the chamber with 100% oxygen gas. The oxygen partial pressure differential gradient was maximized with the use of 100% oxygen. The samples were kept in their respective conditions until the headspace and dissolved oxygen levels reached ~20% which was considered the termination point. An oxygen measurement system was utilized to determine the oxygen level at both locations. Triplicate samples were used per packaging type and treatment conditions.

TABLE 1

| | PET | HDPE | PP |
|---|---|---|---|
| UASLT pressure 1 (psig) | 10 | 10 | 15 |
| UASLT pressure 2 (psig) | 15 | 15 | 20 |
| UASLT pressure 3 (psig) | 20 | 20 | 30 |
| Volume of bottle (mL) | 276 | 150 | 59 |
| Total thickness (mm) | 0.45 | 0.91 | 1.21 |
| Total surface area (m²) | 0.022 | 0.026 | 0.013 |
| Thickness of EVOH layer (mm) | 0 | 0.02 | 0.05 |

Table 1 summarizes the experimental conditions and details of packaging materials for the oxygen diffusion study. To account for the difference in the surface area of the bottles, the oxygen transmission rate (OTR) was calculated according to equation 1, where Q is the amount of oxygen (cm³) measured experimentally, A is the surface area (m²) and t is the time (day)

$$O TR = \frac{Q}{At} \qquad ( Eq . 1 )$$

Statistical analysis was performed. Means of different treatments were analyzed and significant differences between the means were determined using Tukey's pairwise comparison at α=0.05. T-test was also conducted to analyze the difference in means of headspace and dissolved OTR at α=0.05.

Diffusion of oxygen across the polymeric packages was assumed to follow Fick's Law of Diffusion. Fick's diffusion model governing time-dependent diffusion in 2D axisymmetric geometry as shown in equation 2 was solved numerically to estimate the concentration of oxygen in the bottles over time. In equation 2, O₂ (mol·m⁻³) is the molar concentration of oxygen at time t, D (m²s⁻¹) is the diffusion coefficient of the gas, r is the radial dimension, and z is the axial dimension. The boundary conditions applied to the simulation were 1) migration of gases occurred through all sides of the bottles, 2) the temperature was constant at 40° C., and 3) the oxygen concentration at the boundary was maintained at 79.13 mol·m⁻³, 52.97 mol·m⁻³, 39.57 mol·m⁻³, 26.37 mol·m⁻³ and 7.98 mol·m⁻³ to represent 30 psig, 20 psig, 15 psig, 10 psig and 1 atm, respectively. The subscripts h and d indicate headspace and dissolved oxygen, respectively in equations (3.3)-(3.5).

$$\frac{1}{r} \frac{\partial }{\partial r} [ D r \frac{\partial {O}_{2}}{\partial r} ] + \frac{\partial }{\partial z} [ D \frac{\partial {O}_{2}}{\partial z} ] = \frac{\partial {O}_{2}}{\partial t} for < r \leq R , 0 < z \leq Z , t > 0 \qquad ( Eq . 2 )$$

$$and$$

$$C = f ( t )$$

The boundary conditions were,

$$\frac{\partial {C}_{d}}{\partial r} ( R , z , t ) = {O}_{2 , d} ( t ) , \frac{\partial {O}_{2 , d}}{\partial z} ( r , 0 , t ) = {O}_{2 , d} ( t ) , \frac{\partial {O}_{2 , d}}{\partial z} ( r , Z , t ) = {O}_{2 , d} ( t ) \qquad ( Eq . 3 )$$

$$\frac{\partial {C}_{d}}{\partial r} ( R , z , t ) = {O}_{2 , d} ( t ) , \frac{\partial {O}_{2 d}}{\partial z} ( r , 0 , t ) = {O}_{2 , d} ( t ) , \frac{\partial {O}_{2 , d}}{\partial z} ( r , Z , t ) = {O}_{2 , d} ( t ) \qquad ( Eq . 4 )$$

The initial conditions were,

$${O}_{2 , h} ( r , z , 0 ) = {O}_{2 , h , 0} and {O}_{2 , d} ( ( r , z , 0 ) = {O}_{2 , d , 0} \qquad ( Eq . 5 )$$

The experimental data of headspace and dissolved oxygen over time was used to determine the diffusion coefficient using sequential estimation. Data obtained from modelling was used to provide predicted data for the estimation. The scaled sensitivity coefficient (SSC), which indicates the changes in the response variable (oxygen concentration) caused by perturbation in the estimated parameter (diffusion coefficient) is given in equation 6.

$${X}_{i}^{′} = {D}_{i} {( \frac{\partial C}{\partial D} )}_{i} \qquad ( Eq . 6 )$$

The calculation of the percentage of SSC for each estimation is given in equation 7.

$${X}^{′} ( \% ) = \frac{{X}_{max}^{′}}{{\text{?}}_{max} - {\text{?}}_{min}} \times 100 \% \qquad ( Eq . 7 )$$

The sequential estimation of diffusion coefficient was developed using matrix inversion lemma based on the Gauss Minimization method with an appropriate initial guess. Maximum a posteriori (MAP) was used to derive the mathematical form of non-linear sequential estimation for multiple parameters. The Gauss minimization is expressed as:

$$S = {[ Y - \text{?} ( β ) ]}^{′} W [ Y - \text{?} ( β ) ] + {[ μ - β ]}^{′} U [ μ - β ] \qquad ( Eq . 8 )$$

where Y is the experimental response variable, Ŷ is the predicted response, μ is the prior information of parameter vector β, W is the inverse of the covariance matrix of errors, and U is the inverse covariance matrix of parameters. β was solved and reported as the estimated diffusion coefficient. The iterative sequential procedure is given in equations 9-14.

$${A}_{i + 1} = {P}_{i} {X}_{i + 1}^{′} \qquad ( Eq . 9 )$$

$${Δ}_{i + 1} = {ϕ}_{i + 1} + {X}_{i + 1} {A}_{i + 1} \qquad ( Eq . 10 )$$

$${K}_{i + 1} = {A}_{i + 1} {Δ}_{i + 1}^{- 1} \qquad ( Eq . 11 )$$

$${e}_{i + 1} = {Y}_{i + {1}^{-}} {\text{?}}_{i + 1} \qquad ( Eq . 12 )$$

$${b}_{i + 1}^{*} = {b}_{i}^{*} + {K}_{i + 1} [ {e}_{i + 1} - {X}_{i + 1} ( {b}_{i}^{*} - b ) ] \qquad ( Eq . 13 )$$

$${P}_{i + 1} = {P}_{i} - {K}_{i + 1} {X}_{i + 1} {P}_{i} \qquad ( Eq . 14 )$$

where A is the inversion matrix, X is the sensitivity matrix, Δ is the sequential delta, K is the gain matrix, e is the error vector, b is the parameter index, P is the covariance matrix, and i is the iteration index. The stopping criteria for this process is given in equation 15.

$$\frac{\vert {b}_{j}^{k + 1} - {b}_{j}^{k} \vert}{\vert {b}_{j}^{k} + {δ}_{1} \vert} < δ \qquad ( Eq . 15 )$$

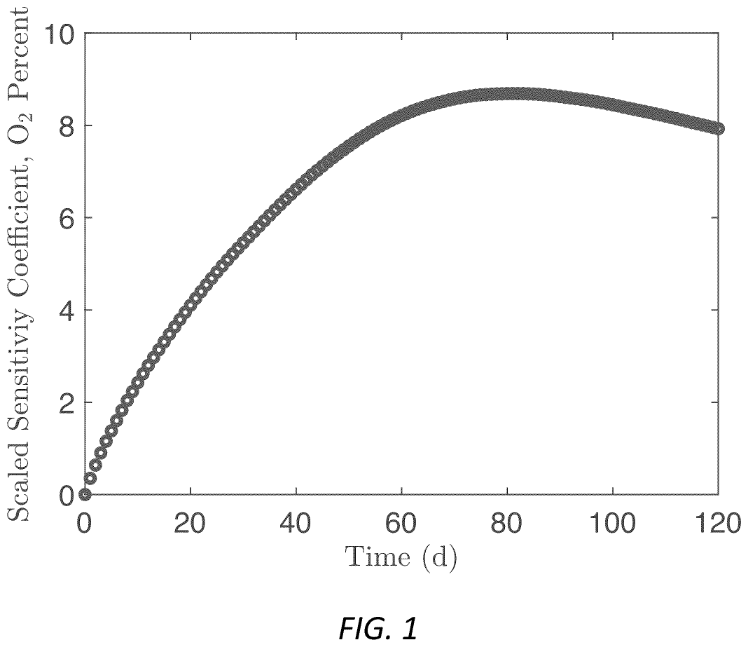

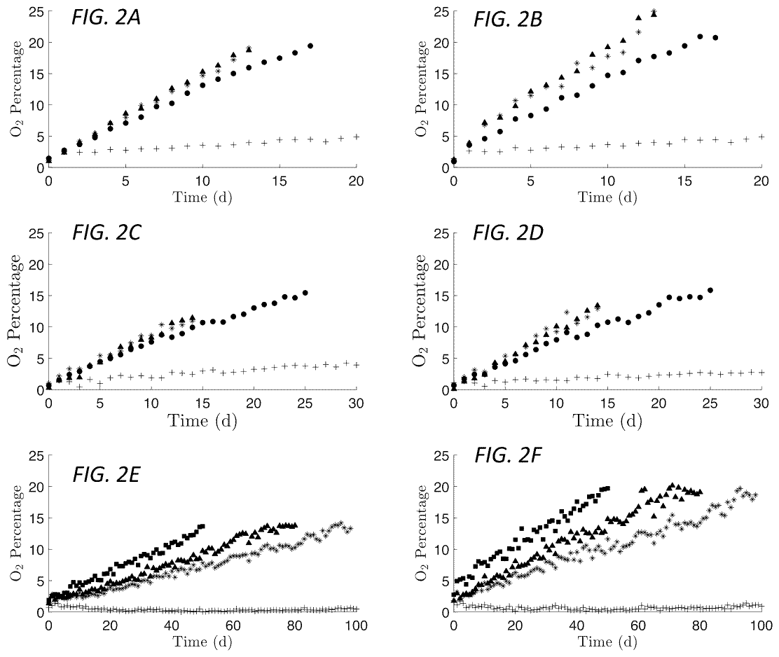

The impact of applying various oxygen pressures to induce oxygen migration in PET, HDPE and PP was investigated. The scaled sensitivity of diffusion coefficient toward changes in oxygen concentration over time was analyzed prior to the experiment as shown in FIG. 1. The increase in the headspace and dissolved oxygen levels in PET bottles containing distilled water are shown in FIG. 2A and FIG. 2B. These samples were kept at various pressure levels of UASLT (10, 15, and 20 psig) with the temperature maintained at 40° C. The comparison with the control samples at the ASLT condition indicates that the oxygen diffusion at the UASLT condition increased significantly with additional pressure. The levels in ASLT samples were still under 5% for both headspace and dissolved oxygen whereas the oxygen amount in samples kept under additional oxygen pressure rose to ambient oxygen concentration (~21%) within 14 to 17 days.

The rate of oxygen transfer into the bottles was dependent on the level of applied pressure. Among the various pressure levels used, the oxygen transfer was fastest at UASLT 20 psig, followed by UASLT 15 psig, UASLT 10 psig and ASLT (FIG. 2A and FIG. 2B). The oxygen increment in UASLT 20 psig and UASLT 15 psig were similar, while the difference in oxygen transfer between UASLT 20 psig and UASLT 10 psig was more prominent. The difference in oxygen accumulation between samples kept at UASLT 15 psig and UASLT 10 psig was slightly larger, compared to the difference between UASLT 20 psig and UASLT 15 psig samples. The positive impact of the pressure was also seen with the OTR in the PET as shown in Table 2 for both the headspace and dissolved oxygen.

TABLE 2
Oxygen Transmission rate (cm³ (STP) · m⁻² · day⁻¹)

| | | Dissolved |
|---|---|---|
| PET | Headspace Oxygen | Oxygen |
| UASLT 20 psig | 152.6 ± 0.93ª | 198.8 ± 1.91ª |
| UASLT 15 psig | 153.9 ± 0.36ª | 187.8 ± 0.20^{b} |
| UASLT 10 psig | 112.9 ± 0.20^{b} | 117.8 ± 0.37^{c} |
| ASLT | 17.8 ± 0.06^{c} | 16.4 ± 0.04^{d} |
| | | Dissolved |
| HDPE | Headspace Oxygen | Oxygen |
| UASLT 20 psig | 42.7 ± 0.47ª | 48.7 ± 0.30ª |
| UASLT 15 psig | 40.0 ± 0.03^{b} | 44.8 ± 0.06^{b} |
| UASLT 10 psig | 32.6 ± 0.17^{c} | 32.6 ± 0.13^{c} |
| ASLT | 6.0 ± 0.04^{d} | 4.5 ± 0.04^{d} |
| | | Dissolved |
| PP | Headspace Oxygen | Oxygen |
| UASLT 30 psig | 15.8 ± 0.05ª | 22.6 ± 0.06ª |
| UASLT 20 psig | 10.0 ± 0.05^{b} | 13.6 ± 0.05^{b} |
| UASLT 15 psig | 7.9 ± 0.05^{c} | 10.9 ± 0.03^{c} |
| ASLT | 0.3 ± 0.01^{d} | 0.5 ± 0.02^{d} |

Table 2 summarizes the oxygen transmission rate (OTR) of headspace and dissolved oxygen in PET, HDPE and PP bottles. The OTR values of the ASLT sample were 16.4 to 17.8 cm³ (STP)·m⁻²·day⁻¹ while the UASLT samples had significantly higher OTR values that were in the range of 112.8 to 1988 cm³ (STP)·m⁻²·day⁻¹.

The increases in the headspace and dissolved oxygen levels in HDPE bottles containing distilled water are shown in FIG. 2C and FIG. 2D. Similar to FIG. 2A and FIG. 2B, samples kept under UASLT conditions exhibited a considerable accumulation in the headspace and dissolved oxygen over time compared to ASLT. The difference in the amount of oxygen accumulated under the three UASLT conditions was not large in magnitude. The intervals of pressure used in the study are probably narrow for HDPE material to demonstrate a substantial difference in oxygen accumulation. Nevertheless, all samples kept at UASLT conditions showed significant increases in oxygen levels compared to ASLT, regardless of the pressure level. All HDPE bottles kept in UASLT condition cracked after a certain period of time due to elevated pressure. Hence, the oxygen levels did not reach ~21% as seen in the PET bottles. This indicates the pressure levels have to be chosen appropriately for each packaging material to avoid significant deformation to the bottles. Similar to PET bottles, the OTR values for HDPE bottles kept at UASLT condition were higher compared to ASLT. The range of OTR values for HDPE bottles was slightly lower than PET in the range of 4.5 to 48.7 cm³ (STP)·m⁻²·day⁻¹ (Table 2). These OTR values were significantly lower compared to the OTR values of PET despite having a larger surface area due to the presence of an EVOH layer that increases resistance toward oxygen transmission.

FIG. 2E and FIG. 2F depict the increase in the headspace and dissolved oxygen levels in PP containers. These bottles contained a thicker EVOH layer sandwiched between the PP layers to strengthen the oxygen barrier properties. Due to increased barriers, oxygen transfer in these bottles was relatively slow compared to PET (no EVOH layer) and HDPE (thinner EVOH layer). The time taken for oxygen accumulation to reach the termination point in samples kept at UASLT 20 psig was 14 days for PET and HDPE, and 79 days for PP bottles with the EVOH layer. Although these samples were resistant to oxygen, the impact of added oxygen pressure was noticeable compared to the ASLT (control) samples. The effect of different pressure levels on the amount of oxygen transfer was also notable in these samples. The time taken for oxygen accumulation to reach the termination point was 50, 79 and 98 days for UASLT 30 psig, UASLT 20 psig and UASLT 15 psig, respectively. For all three pressure levels, the time for PP samples to reach termination was longer (50 to 98 days) compared to other samples while PET was in the range of 14 to 18 days and HDPE was in the range of 14 to 25 days. In PET and HDPE, the times taken to reach the termination points between UASLT 15 and UASLT 20 psig were close to each other (FIGS. 2A, B, C, and D), as opposed to PP (FIG. E and FIG. F) at the same pressure level. This is mainly resulting from the presence of a thicker EVOH layer of PP compared to the EVOH layer in the HDPE bottles (Table 1), providing an additional barrier for oxygen transmission. The OTR values of PP bottles were in the range of 0.3 to 22.6 cm³ (STP)·m⁻²·day⁻¹ as shown in Table 2.

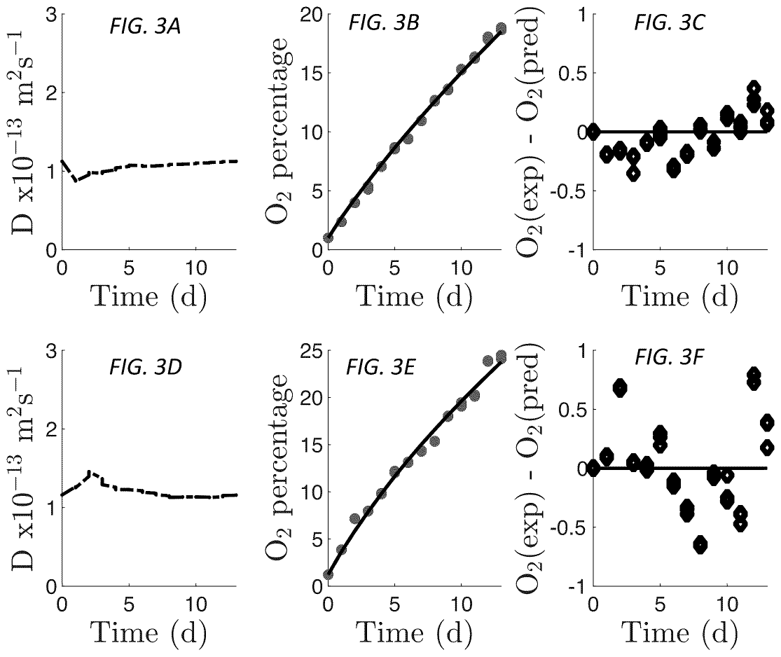

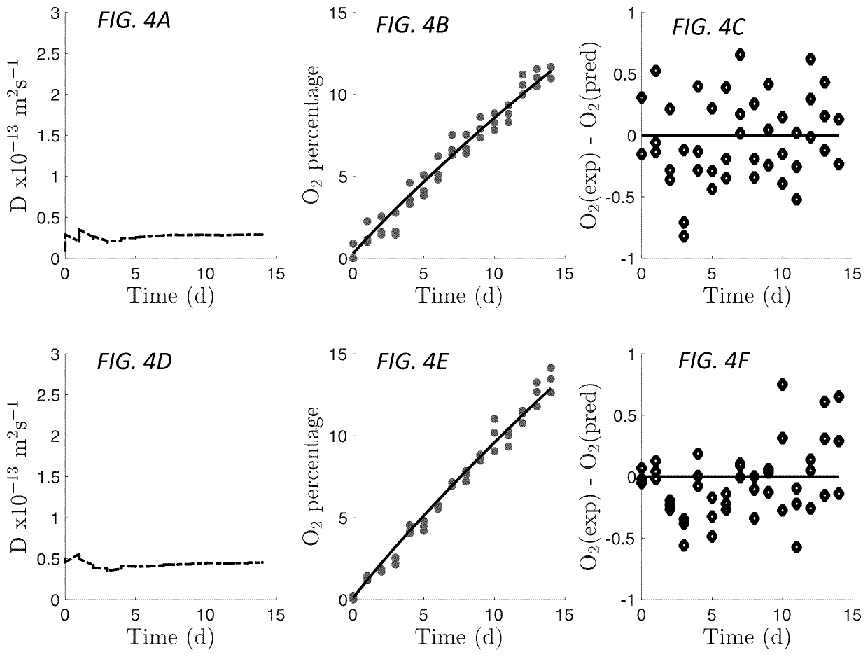

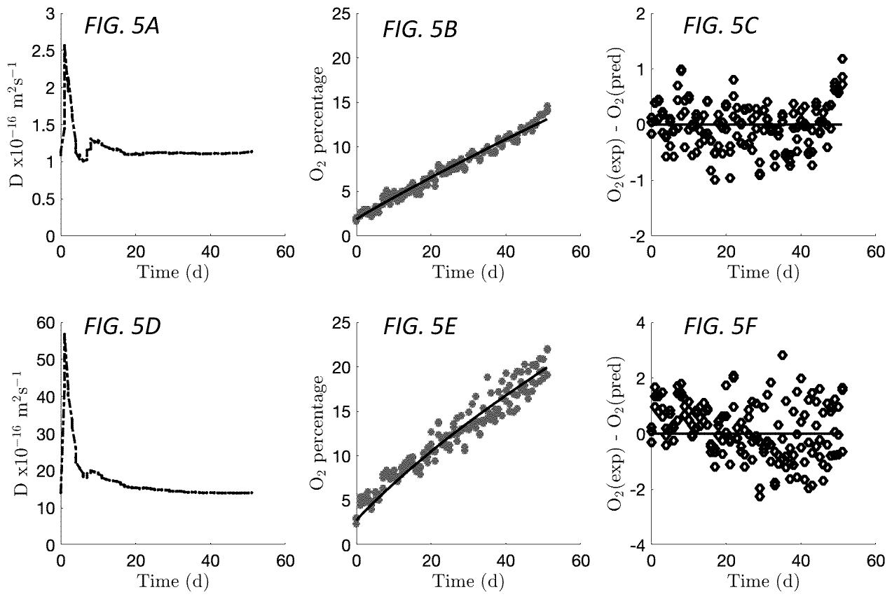

The accumulation of headspace and dissolved oxygen data (FIGS. 2A through 2F) were used to determine the diffusion coefficient of oxygen using the parameter estimation technique as detailed materials and methods. Examples of the estimation plots and residual analysis are presented in FIGS. 3A-3F, FIGS. 4A-4F, and FIGS. 5A-5F for PET, HDPE and PP, respectively. For all samples from different treatments, the sequential estimation arrived at a constant value, indicating that further data collection was not necessary. The residuals were randomly distributed without any patterns.

The parameter estimation results are summarized in Tables 3-5 for the headspace and dissolved oxygen, along with the 95% confidence intervals and root mean square errors (RMSE) of the estimate. The magnitudes of SSC were also reported to assess the reliability of the estimate. Table 3 shows the estimation of the diffusion coefficient of headspace and dissolved oxygen for PET bottles. Overall, the diffusion coefficient was estimated with relatively low RMSE and large magnitudes of SSC for all experiments. As the pressure in the environment increased, the diffusivity of oxygen increased as well for both the headspace and dissolved oxygen. The diffusion coefficients of the samples kept under pressure were in the ranges of 0.86 to 1.11×10⁻¹³ m²s⁻¹ and 0.76 to 1.14×10⁻¹³ m²s⁻¹ for the headspace and dissolved oxygen, respectively. The diffusion coefficients of samples kept in ASLT conditions were lower than samples from UASLT with values around 0.67×10⁻¹³ m²s⁻¹ and 0.50×10⁻¹³ m²s⁻¹ for the headspace and dissolved oxygen, respectively. The RMSE of estimate for ASLT samples was slightly higher than the USALT samples for all pressure levels. An increasing trend was seen in the diffusion coefficient with increasing pressure levels used in the study. The difference in the diffusion coefficient of samples kept at UASLT 20 psig and UASLT 15 psig was not as noticeable as the difference seen between samples kept at UASLT 15 psig and UASLT 10 psig as well as between UASLT 10 psig and UASLT 20 psig. This corresponds to the observations noticed in FIGS. 2A and 2B.

TABLE 3

| | | | | | |
|---|---|---|---|---|---|
| | Diffusion | | | | |
| | coefficient | | | | |
| Treatment | D × 10⁻¹³ (m²s⁻¹) | LCI_{95%} | UCI_{95%} | RMSE | SSC (%) |

Headspace Oxygen

| | | | | | |
|---|---|---|---|---|---|
| ASLT | 0.684 | 0.671 | 0.696 | 0.516 | 71.43 |
| UASLT 10 psig | 0.885 | 0.860 | 0.910 | 0.258 | 83.36 |
| UASLT 15 psig | 1.080 | 1.041 | 1.119 | 0.404 | 82.89 |
| UASLT 20 psig | 1.124 | 1.111 | 1.134 | 0.174 | 83.13 |

Dissolved Oxygen

| | | | | | |
|---|---|---|---|---|---|
| ASLT | 0.507 | 0.506 | 0.508 | 0.752 | 71.43 |
| UASLT 10 psig | 0.783 | 0.757 | 0.809 | 0.325 | 77.25 |
| UASLT 15 psig | 1.092 | 1.045 | 1.139 | 0.590 | 76.66 |
| UASLT 20 psig | 1.161 | 1.137 | 1.185 | 0.383 | 76.24 |

Table 4 summarizes the estimation of the diffusion coefficient of headspace and dissolved oxygen for HDPE bottles. The diffusion coefficient values were in the range of 0.144 to 0.198×10-13 m2s-1 for the samples kept in the ASLT conditions and were in the range of 0.228 to 0.456×10-13 m2s-1 for the UASLT samples. Similar to what was observed in the PET samples, the diffusion coefficients were considerably different for ASLT and UASLT samples. UASLT samples had enhanced diffusion that followed the level of the pressure applied.

TABLE 4

| | | | | | |
|---|---|---|---|---|---|
| | Diffusion | | | | |
| | coefficient | | | | |
| Treatment | D × 10⁻¹³ (m²s⁻¹) | LCI_{95%} | UCI_{95%} | RMSE | SSC (%) |

Headspace Oxygen

| | | | | | |
|---|---|---|---|---|---|
| ASLT | 0.198 | 0.192 | 0.203 | 0.697 | 75.93 |
| UASLT 10 psig | 0.228 | 0.225 | 0.232 | 0.144 | 81.54 |
| UASLT 15 psig | 0.267 | 0.255 | 0.228 | 0.315 | 86.36 |
| UASLT 20 psig | 0.287 | 0.278 | 0.296 | 0.338 | 85.38 |

Dissolved Oxygen

| | | | | | |
|---|---|---|---|---|---|
| ASLT | 0.144 | 0.133 | 0.154 | 1.256 | 85.00 |
| UASLT 10 psig | 0.316 | 0.306 | 0.326 | 0.396 | 83.89 |
| UASLT 15 psig | 0.442 | 0.423 | 0.461 | 0.376 | 86.57 |
| UASLT 20 psig | 0.456 | 0.445 | 0.467 | 0.290 | 86.26 |

TABLE 5

| | | | | | |
|---|---|---|---|---|---|
| | Diffusion | | | | |
| | coefficient | | | | |
| Treatment | D × 10⁻¹⁶ (m²s⁻¹) | LCI_{95%} | UCI_{95%} | RMSE | SSC (%) |

Headspace Oxygen

| | | | | | |
|---|---|---|---|---|---|
| UASLT 15 psig | 0.585 | 0.5776 | 0.5922 | 0.2649 | 88.11 |
| UASLT 20 psig | 0.809 | 0.7888 | 0.815 | 0.4498 | 87.36 |
| UASLT 30 psig | 1.139 | 1.1229 | 1.1541 | 0.418 | 88.64 |

Dissolved Oxygen

| | | | | | |
|---|---|---|---|---|---|
| UASLT 15 psig | 6.498 | 6.396 | 6.6001 | 0.4963 | 90.66 |
| UASLT 20 psig | 9.145 | 9.0251 | 9.2648 | 0.5848 | 93.73 |
| UASLT 30 psig | 14.093 | 13.7997 | 14.3856 | 1.011 | 89.09 |

Table 5 summarizes estimation of the diffusion coefficient of headspace and dissolved oxygen for PP bottles. (Diffusion coefficient from ASLT is not available since no significant increase in oxygen was seen over time.) The oxygen diffusion coefficients in PP samples were kept at UASLT conditions. Diffusion coefficient estimation for ASLT samples was not performed as there was no significant increase in the headspace and dissolved oxygen levels over time. UASLT samples showed increasing diffusion coefficients according to the pressure level applied, similar to what was observed with PET and HDPE samples. All UASLT samples of PP appeared to have a smaller diffusion coefficient for both headspace and dissolved oxygen compared to the PET and HDPE samples. Their values were in the range of 0.58 to 1.14×10-16 m2s-1 and 6.49 to 14.09×10-16 m2s-1 for headspace and dissolved oxygen, respectively. A major difference was seen between the headspace and dissolved oxygen diffusion coefficients in PP compared to PET and HDPE samples.

The impact of additional pressure on the oxygen diffusion coefficient was significant in all three different packaging materials. These materials inherently have different barrier properties against oxygen. Based on the diffusion coefficient values presented in Tables 3-5, the order of the materials from least to most permeable to oxygen was, PP with EVOH layer<HDPE with EVOH layer<PET. The oxygen diffusion coefficient values reported in this study were consistent with values reported in the literature for PET material. The diffusion coefficient of oxygen in semi-crystalline PET was 3.3×10⁻¹³ m²s⁻¹, and in amorphous PET at 25° C. and 40° C. were 5.6×10⁻¹³ m²s⁻¹ and 11.6×10⁻¹³ m²s⁻¹, respectively. Without the presence of the EVOH layer, PP and HDPE are more permeable than PET. However, the oxygen barrier property is improved in the presence of the EVOH layer. Previous studies revealed that the diffusion coefficients of oxygen in HDPE and PP without the EVOH layer are in the range of 1.2 to 4.3×10⁻¹¹ m²s⁻¹ and 2.4×10⁻¹¹ to 2.5×10⁻¹³ m²s⁻¹, respectively. Depending on the thickness and the crystallinity of the EVOH layer, the permeability decreases up to 3 to 5 orders of magnitude when EVOH is added to HDPE layers and 2-3 magnitude for PP materials. EVOH is a semicrystalline copolymer of ethylene and vinyl alcohol monomers and has high resistance to oxygen. The vinyl alcohol units provide the gas barrier properties attributed to the presence of inter- and intra-molecular bonding by the hydroxyl groups, but it leads to poor barriers against moisture. The presence of water plasticizes the film and makes it more swollen by occupying the space inside the polymer which results in increased oxygen permeability. As the EVOH was protected from moisture by layers of PP and HDPE, the oxygen permeability was low (Table 4 and Table 5). The pronounced effect of elevated oxygen pressure on the diffusivity of oxygen on materials containing EVOH layer substantiates the effectiveness of UASLT even for resistant packaging.

In all UASLT treatments, one common observation noted was that the dissolved oxygen level was always higher than the headspace oxygen level when the samples were placed in the pressurized environment (FIGS. 2A through 2F). Such observation was not made in the control samples with both headspace and dissolved oxygen remaining at relatively similar levels with a slow increase over time. It appears that the enhanced diffusion as a result of pressure is more prominent when the walls of the bottle are in contact with a liquid. This indicates that the presence of liquid material in the bottle increases the rate of oxygen permeation. Oxygen permeation in the headspace is merely governed by the difference in the partial pressure of oxygen inside the bottle and the pressure chamber. On the contrary, the oxygen transfer to the water is driven by the partial pressure gradient as well as the solubility of oxygen in water. Solubilization of oxygen also occurs at the headspace and water interface, leading to a higher oxygen amount in the water compared to the headspace. An equilibrium may be achieved between the headspace and dissolved oxygen if the samples were given enough time. Literatures have reported changes in the polymer structure and a significant impact on the barrier properties in the presence of moisture and other liquids. Water molecules and/or other small molecular mass compounds change the way gas is adsorbed and diffused through the polymer. For some materials, an increase in surrounding moisture can drastically raise the oxygen permeability and change the overall diffusion mechanism due to altered solubility. In amorphous polymers, the water molecules fill the voids within the polymer chains which increases the oxygen solubility, and in semi-crystalline polymers, clusters of water molecules in the polymer disrupt the crystalline region creating additional sites for oxygen to permeate. A combination of reasons explained above could underline the high dissolved oxygen accumulation compared to the headspace amount in all UASLT samples. Since these effects were not seen in ASLT samples, it is most likely that the effects were more pronounced due to the usage of elevated pressure and temperature.

Reports have shown increased oxygen diffusivity at higher pressure levels. Pressure levels used in these studies were between 7 to 4000 psig at room temperature. Some articles also have reported decreasing diffusion coefficient at increasing pressure for glassy polymers due to a plasticizing effect. At high pressure, the contribution of the Langmuir region (capacity for solubilization) to overall permeability is weaker and the gas permeability approaches a constant value associated with simple dissolution transport. For glassy polymers such as PET, the dual-mode is used to describe the gas sorption which consists of both Henry's and Langmuir's terms to account for the physical changes due to glass transition, and non-linear increase in solubility with increasing pressure. For non-glassy polymers, the solubility follows Henry's law. The glass transition of the glassy polymers involved in this study was 80 to 82° C. and 50 to 63° C. for PET and EVOH, respectively. As the current study only utilized elevated pressure from 10 to 30 psig at 40° C. and reported increased oxygen diffusivity, it is highly unlikely for plasticization of the glassy polymers to occur. Given the rapid influx of oxygen under pressure, it is not unreasonable to speculate a slight increase in the polymer flexibility. It is unclear if any significant microscopic changes have occurred in the polymer structure due to the pressure applied.

The use of multiple accelerant factors appears to be important for an effective accelerated shelf-life analysis method for shelf-life determination and prediction for food products with low errors. As demonstrated above, elevated external oxygen pressure can be used as an accelerant along with elevated temperature for rapid shelf-life testing of oxygen-sensitive products in polymeric packaging. The application of high external pressure enhanced the diffusion of oxygen through the food packaging for PET, HDPE and PP materials. The application of inverse problems enabled the determination of the diffusion coefficient of oxygen with minimal errors and small confidence intervals. The diffusion coefficient increased with rising pressure levels. The rapid influx of oxygen in UASLT has the potential to increase the rate of degradation reactions in foods which will significantly reduce the overall shelf-life analysis time. Results from UASLT can be used to better predict the rate of degradation reactions of food at ambient storage conditions.

In view of these data, according to some aspects of the invention, oxygen pressure can be applied to the external environment of hermetically sealed packaged foods to increase the oxygen transfer to the food for inducing rapid degradation at elevated temperatures. The oxygen diffusivity can be manipulated by adjusting the oxygen pressure to ensure the internal environment of the food does not accumulate oxygen above 21%. This new approach provides a new way to decrease the overall time needed to analyze shelf-life that may improve the time to product commercialization for the food industry. The increase in the oxygen transfer with the use of high external oxygen pressure can increase the rate of degradation reactions in food which can lead to a reduction in the overall shelf-life analysis time at 40° C. The multi-accelerant approach that combines oxygen pressure and elevated temperature has the potential to be used for the rapid shelf-life analysis of food products in polymeric packaging.

In the course of developing the present invention, certain additional tests were conducted, which are summarized hereinafter.

A shelf-stable model food fortified with vitamins A, B1, C and D3 was developed to investigate the effect of multiple accelerants on the quality indicators of shelf-stable foods in a polyethylene terephthalate (PET) container. PET bottles filled with model food were placed in a high-pressure (138 kPa) 100% oxygen environment at 40° C. This new process is named as the ultra-accelerated shelf-life test (UASLT). Samples were also subjected to ASLT conditions at 40° C. and control at 22.5° C., both at ambient pressure for comparison. UASLT treatment induced a rapid degradation of 27.1±1.9%, 35.8±1.0%, and 35.4±0.7% in vitamins A, C and D3, respectively, in just 50 days. Slower degradation was observed with samples kept under the ASLT conditions for 105 days with a degradation of 24.0±2.0%, 32.0±3.1% and 25.1±1.5% for vitamin A, C and D3, respectively. The control samples that were studied for 210 days showed 14.9±5.0%, 13.8±2.2% and 10.6%±0.8% degradation in vitamins A, C and D3, respectively. The increase in the ΔE values due to browning in samples kept at the UASLT, ASLT and control conditions were 11.67±0.09, 7.49±0.19 and 2.51±0.11, respectively. The degradation of vitamin B1 was similar across the treatments. The addition of oxygen pressure significantly increased the degradation reaction rates of the vitamins and color due to the rapid influx of oxygen. A mechanistic model that coupled oxygen diffusion and simultaneous vitamin degradation provided a good fit to the experimental data for the UASLT treatment with a rate constant of 0.686, 0.631 and 0.422M⁻¹ day⁻¹ for vitamins C, D3 and A, respectively. The mechanistic model was not suitable to model the vitamin degradations in the ASLT and control samples. From this, it can be seen that elevated external oxygen pressure can be used as an accelerant along with moderate temperatures for rapid shelf-life testing of products in polymeric packaging with two fold reduction in the overall analysis time.

A model food fortified with vitamins A, B1, C and D3 was developed according to the formulation presented in Table 6.

TABLE 6
Formulation of model food

| Ingredient | Percentage (w/w) |
|---|---|
| Water | 57.7% |
| Mango puree | 28.0% |
| Sugar | 9.0% |
| Whey protein | 1.0% |
| Pectin stabilizer | 0.3% |
| Sunflower lecithin | 0.2% |
| Citric acid | 0.3% |
| Ascorbic acid (vitamin C) | 1.0% |
| Thiamine hydrochloride (vitamin B1) | 1.0% |
| Retinyl palmitate mix (vitamin A) | 0.5% |
| Cholecalciferol mix (vitamin D3) | 1.0% |

After hydrating the whey protein and pectin stabilizer in water with agitation for 20 min, the rest of the ingredients were added. The pH was verified to be 4.00±0.03. The model food was processed and aseptically packaged. Before introducing the model food, both the UHT and filler units were sterilized. The sterility of the filler head was maintained by having sterile over pressure, keeping the temperature above 60° C. between fill cycles and a spray of 140 ppm chlorine into the filler head. The product was processed at a flow rate of 1 mL/min with a holding time of 36s and a target end of hold tube temperature of 100° C. The process was designed to deliver a lethality of 1 min at a reference temperature of 93.33° C. and a z-value of 8.89° C. The product was cooled down to 24° C. before aseptically filling into a pre-sterilized 1-gallon bag-in-box package. The spout on the bags was sterilized before filling the product.

Bottles filled with the model food were placed in a custom-made high-pressure chamber supplied with 100% oxygen gas at 138 kPa (20 psig) and 40° C., in accordance with certain example aspects of the present invention. This treatment is referred to as the ultra-accelerated shelf-life test (UASLT). Another set of samples was placed in an incubator at 40° C. without additional pressures following the conventional ASLT method. Control samples were placed at room temperature and pressure. All UASLT, ASLT, and control samples were kept in the dark. Samples were removed from the storage condition at pre-determined intervals to quantify the changes in color and vitamin levels over time.

A colorimeter was used to quantify the changes in color in the model food. The color measurements were recorded using the CIELab uniform color space with reference illuminant D65 and a visual angle of 10°. The total color difference, ΔE, was calculated using equation 16 based on the recorded L*, a*, b* values.

$$Δ E = \sqrt{Δ {L}^{* 2} + Δ {a}^{* 2} + Δ {b}^{2}} \qquad ( Eq . 16 )$$

The color of the aseptically processed model food before the storage treatment was used as the reference.

Extraction of vitamin A (retinyl palmitate) and D3 (cholecalciferol) was performed simultaneously according to AOAC Official Method 2012.09. Simultaneous extraction of vitamin B1 (thiamin hydrochloride) and C (ascorbic acid) was performed. Calibration curves were developed using retinyl palmitate, cholecalciferol, thiamin hydrochloride, and ascorbic acid as standards for the quantification of the remaining vitamins.

A mechanistic model of oxygen diffusion and simultaneous vitamin degradation was used. The oxygen driven degradation of vitamins can be described as bimolecular reactions as shown in equations 17-19.

$$Vit C + {O}_{2} {\to }^{{k}_{C}} product 1 \qquad ( Eq . 17 )$$

$$Vit D 3 + {O}_{2} {\to }^{{k}_{D 3}} product 2 \qquad ( Eq . 18 )$$

$$Vit A + {O}_{2} {\to }^{{k}_{A}} product 3 \qquad ( Eq . 19 )$$

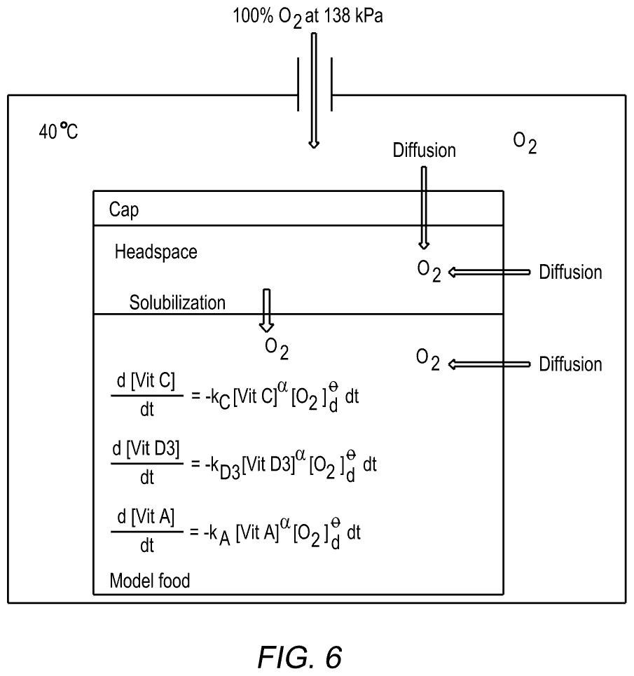

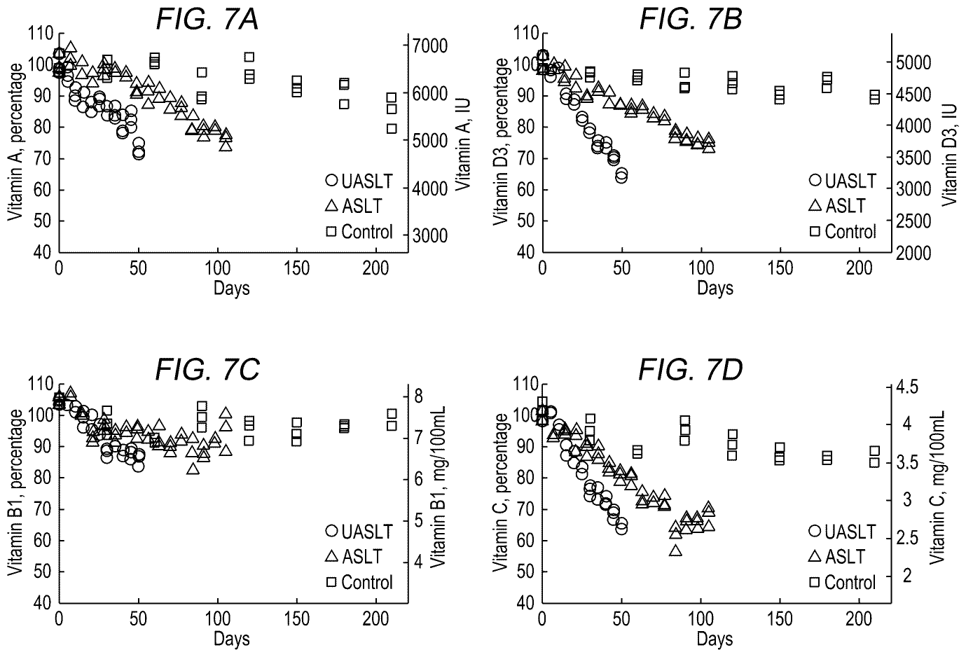

The rate law describing these bimolecular reactions are shown in equations 20-23. The order of reaction with respect to the reactants, α and θ was assumed to be 1. This makes the reactions represented in equations 20-22 follow the second-order kinetic. Equations 20-22 show that the rate change of vitamins is dependent on the initial concentration of vitamin, the amount of oxygen available for the reaction and the corresponding rate of reaction, (M⁻¹·day⁻¹). The amount of oxygen participating in the reaction is dependent on the initial amount of oxygen present in the hermetically sealed food as well as the amount of oxygen diffusing into the product through the packaging materials as shown in equations 23-26. The mechanistic model of oxygen diffusion and simultaneous degradation of vitamin C, D3 and A in model foods kept at the UASLT condition are presented in FIG. 6. A similar approach was also used to model the vitamin degradation in model foods kept at the ASLT and control condition using atmospheric oxygen concentration at the boundary of the PET bottles. Degradation of vitamin B1 was not modeled as no significant reduction in the concentration was seen in any treatment FIG. 7-C.

$$\frac{d [ VitC ]}{dt} = - {{{k}_{C} [ VitC ]}^{α} [ {O}_{2} ]}_{d}^{θ} dt \qquad ( Eq . 20 )$$

$$\frac{d [ Vit D 3 ]}{dt} = - {{{k}_{D 3} [ VitD 3 ]}^{α} [ {O}_{2} ]}_{d}^{θ} dt \qquad ( Eq . 21 )$$

$$\frac{d [ Vit A ]}{dt} = - {{{k}_{A} [ VitA ]}^{α} [ {O}_{2} ]}_{d}^{θ} dt \qquad ( Eq . 22 )$$

The amount of oxygen diffused into the bottle at time, t:

$$\frac{\partial {O}_{2 , A}}{\partial t} = \frac{1}{r} \frac{\partial }{\partial r} [ Dr \frac{\partial {O}_{2 , d}}{\partial r} ] + \frac{\partial }{\partial z} [ D \frac{\partial {O}_{2 , d}}{\partial z} ] \qquad ( Eq . 24 )$$

For 0<r<R, 0<z<Z, t>0 and O₂=f(t). The boundary conditions were

$$\frac{\partial {O}_{2 , d}}{\partial r} ( R , z , t ) = {O}_{2 , d} ( t ) , \frac{\partial {O}_{2 , d}}{\partial z} ( r , 0 , t ) = {O}_{2 , d} ( t ) , \frac{\partial {O}_{2 , d}}{\partial z} ( r , Z , t ) = {O}_{2 , d} ( t ) , \qquad ( Eq . 25 )$$

and initial conditions were

$${O}_{2 , d} ( r , z , 0 ) = {O}_{2 , d , 0} \qquad ( Eq . 26 )$$

Differential equations 20-23 were solved simultaneously by a method that requires the absolute error between 5^{th}- and 4^{th}-order methods to be below 10⁻⁶.

Diffusion of oxygen across the polymeric packages was assumed to follow Fick's Law of Diffusion. Equation 24 was solved numerically to estimate the concentration of oxygen in the bottles over time. In equation 24, O₂ (mol·m⁻³) is the molar concentration of oxygen at time t, D (m²s⁻¹) is the diffusion coefficient of the gas, r is the radial dimension, and z is the axial dimension. It was assumed that the migration of gases occurred through all sides of the bottles, and the oxygen concentration at the boundary was maintained throughout the experiment duration. The sequential estimation of D (m²s⁻¹) was developed using a matrix inversion lemma based on the Gauss Minimization method with an appropriate initial guess. Maximum a posteriori (MAP) was used to derive the mathematical form of non-linear sequential estimation for multiple parameters. The Gauss minimization is expressed as:

$$S = [ Y - Y ( β ) ] W [ Y - Y ( β ) ] + [ μ - β ] U [ μ - β ] \qquad ( Eq . 27 )$$

where Y is the experimental response variable, Ŷ is the predicted response, μ is the prior information of parameter vector β, W is the inverse of the covariance matrix of errors, and U is the inverse covariance matrix of parameters. β was solved and reported as the estimated diffusion coefficient.

The impact of added oxygen pressure at 40° C. (UASLT) on the degradation of vitamins and changes in color in the model food were investigated and compared with the conventional ASLT method and control. Both the UASLT (138 kPa) and ASLT (0.21 kPa) treatments were conducted at the same temperature 40° C. with different oxygen pressure while the ASLT and the control (22.5° C.) conditions were of the atmospheric pressure. A comparison between these three conditions shows the impact of oxygen pressure as an accelerant at elevated temperatures.

FIGS. 7A through 7D show the degradation of vitamins A, B1, C and D3 in model food kept under UASLT, ASLT and control conditions. The vitamins in the model food kept under UASLT conditions showed rapid degradation over time. A degradation of 27.1±1.9%, 13.9±2.1%, 35.8±1.0%, and 35.4±0.7% was seen in vitamins A, B1, C and D3, respectively in just 50 days. Slower degradation was observed with samples kept under ASLT conditions that needed at least 105 days to reach a similar level of degradation of vitamin A (24.0±2.0%) and vitamin C (32.0±3.1%). Degradation of vitamin D3 in the samples kept under ASLT conditions only reached 25.1±1.5% in 105 days and needs more time to reach a similar level as seen in the samples kept under the UASLT conditions. The vitamin B1 degradation in samples kept at the ASLT conditions was only 4.9±6.1%. The control samples that were studied for 210 days showed 14.9±5.0%, 2.0±2.2%, 13.8±2.2% and 10.6%+0.8% degradation in vitamins A, B1, C and D3, respectively. The temperature and the oxygen pressure applied has no effect on the degradation of vitamin B1. Since the degradation in vitamin B1 was not sufficient for kinetic modeling, it was omitted from further analysis.

All samples were flushed with nitrogen prior to sealing to ensure the residual oxygen at the beginning of the storage study was below 1%. Samples kept under the UASLT condition received a continuous supply of oxygen diffusing through the hermetically sealed PET bottles from the high external oxygen environment and underwent a rapid degradation in vitamins as shown in FIGS. 7A through 7D. The oxygen partial pressure differential gradient was increased by placing the PET bottle under a pressurized (20 psig) environment supplied with 100% oxygen. At this condition, the oxygen partial pressure outside the package was increased, which resulted in a larger pressure differential gradient. The degradation reactions in the model food proceeded rapidly by receiving a supply of oxygen at a rate of 199 cm³ (STP) per day (in accordance with the experiments discussed previously herein). At ASLT condition, the oxygen transfer rate was 16 cm³ (STP) per day indicating that more than 10 times more oxygen was supplied to the samples kept under UASLT condition (in accordance with the experiments discussed previously herein).

In samples kept at the ASLT and control conditions, the degradation reaction was driven by any residual oxygen present initially and then followed by anaerobic degradation in the absence of oxygen while utilizing the oxygen diffusing through the bottles slowly. The diffusivity of oxygen in the ASLT and control conditions were of 0.507×10⁻¹³ m²s⁻¹ (in accordance with the experiments discussed previously herein) and 0.109×10⁻¹³ m²s⁻¹, respectively. Degradation of vitamins C and D3 can proceed without the presence of oxygen which is known as the anaerobic degradation but occurs at a slower rate. The degradation of vitamins in the ASLT and control samples was not entirely anaerobic since there was evidence of oxygen diffusion over time. A lag in degradation (an induction phase) was observed with the degradation of vitamin A in samples kept under ASLT conditions. This may be due to the presence of vitamin C acting as an antioxidant where the presence of vitamin C has been shown to slow down the degradation of vitamin A. The degradation seen in the samples kept at the ASLT condition was significantly higher than what was seen in the control sample, owing to the elevated temperature which is known to increase the rate of the degradation reactions as well as the increased oxygen diffusion.

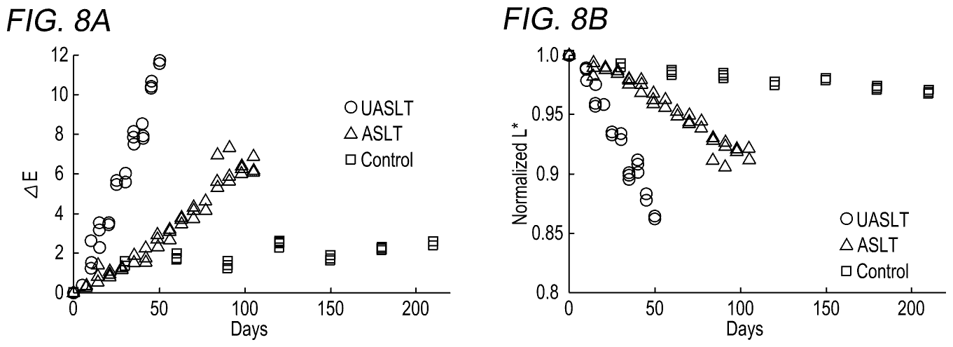

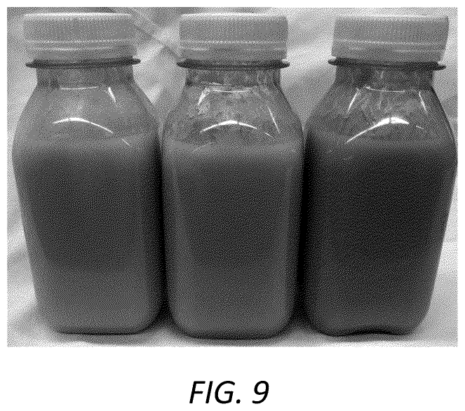

The overall color change for samples kept under UASLT, ASLT, and control conditions is shown in FIGS. 8A and 8B. FIG. 9 illustrates browning of the model food kept at the control (left), ASLT (middle), and UASLT (right) conditions for 50 days. A rapid increase in the ΔE values (11.67±0.09) due to browning was noted with samples kept at UASLT conditions for 50 days compared to the samples kept at ASLT and control conditions. The ΔE values of samples were kept at ASLT and control conditions were 7.49±0.19 and 2.51±0.11 in 105 and 210 days, respectively. The extent of browning in the model food kept at the control, ASLT, and UASLT conditions for 50 days are shown in FIGS. 8A and 8B. The changes in the color of the sample were primarily due to the decrease in the L* values followed by a small decrease in the b* value. The positive a* value which indicates the redness remained unchanged for all samples throughout the study. Since the changes in the b* were very small, only L* values are reported in FIG. 8B. The L* values and positive b* values indicate the lightness and the yellowness of the samples, respectively. The L* and b* decreased over time due to an increase in the brown color or darkness and a decrease in the yellowness, respectively. A similar observation was reported for the browning of orange juices during storage studies. Rapid degradation of vitamin C in samples kept at the UASLT condition most likely led to the formation of furfural which may undergo polymerization or combine with amino acids to form brown melanoidin pigments. This is consistent with the extensive browning observed with the samples kept at the UASLT conditions.

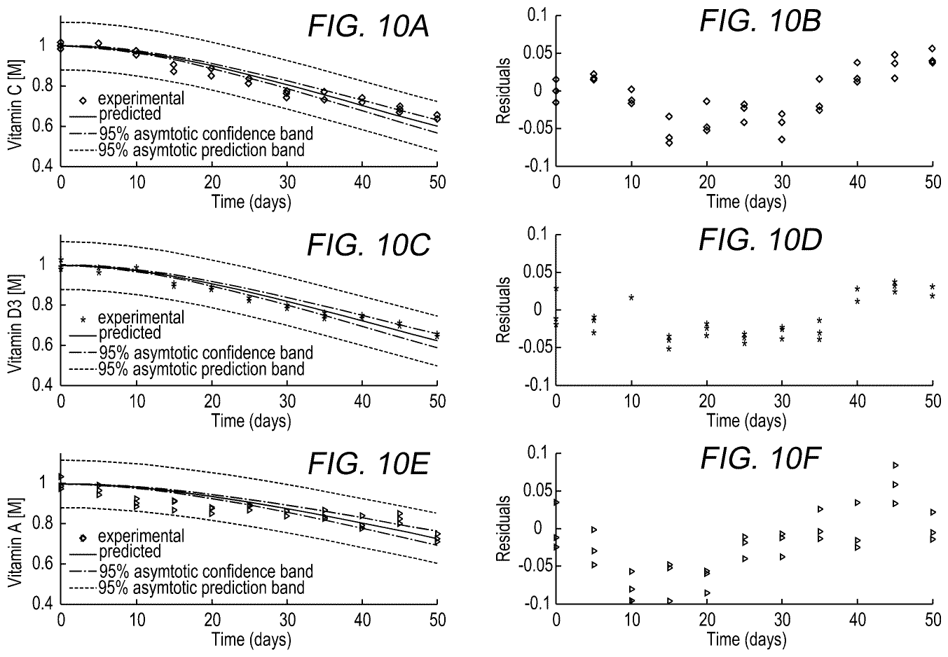

FIGS. 10A through 10F show the degradation of vitamins C, D3, and A in samples kept at the UASLT condition represented with the proposed mechanistic model along with the 95% asymptotic confidence band, 95% asymptotic prediction band and the distribution of the residuals. Overall, the model provided a good fit for the experimental data and the residuals do not appear to violate the standard statistical assumption. The estimates of rate constants k_{C}, k_{D3} and k_{A} for vitamins C, D3 and A along with its 95% confidence interval, standard error, the relative error between parameters and RMSE for samples kept at UASLT, ASLT and control are presented in Table 7.

TABLE 7

| | | k (M⁻¹ · | Standard | Relative | | |
|---|---|---|---|---|---|---|
| | | day⁻¹) | error | error | CI_{95%} | RMSE |
| UASLT | | | | | | |
| Vitamin C | k_{C} | 0.686 | 0.35 | 50.5 | 0.611 ± 0.761 | 0.038 |
| Vitamin | k_{D3} | | | | | |
| D3 | | 0.631 | 0.25 | 39.9 | 0.577 ± 0.685 | |
| Vitamin A | k_{A} | 0.422 | 0.22 | 52.1 | 0.375 ± 0.470 | |
| ASLT | | | | | | |
| Vitamin C | k_{C} | 17.3 | 2.82 | 16.4 | −30.4 ± 64.9 | 0.039 |
| Vitamin | k_{D3} | | | | | |
| D3 | | 13.6 | 2.75 | 20.2 | −23.1 ± 50.3 | |
| Vitamin A | k_{A} | 10.2 | 2.70 | 26.6 | −17.4 ± 37.7 | |
| Control | | | | | | |
| Vitamin C | k_{C} | 89.9 | 3.16 | 3.5 | −142.6 ± 322.6 | 0.037 |
| Vitamin | k_{D3} | | | | | |
| D3 | | 50.0 | 3.16 | 6.3 | −80.4 ± 180.4 | |
| Vitamin A | k_{A} | 60.0 | 3.16 | 5.3 | −96.1 ± 216.1 | |

Table 7 shows that the estimated parameter for UASLT was in the range of 0.422 to 0.686 (M⁻¹·day⁻¹) with tight confidence intervals and low errors. The correlation coefficient matrix of the parameters (Table 8) shows a minimal correlation between the parameters which indicates that the parameters can be estimated uniquely and simultaneously for samples kept at UASLT conditions.

TABLE 8

| | | k_{C} | k_{D3} | k_{A} |
|---|---|---|---|---|
| UASLT | | | | |
| Vitamin C | k_{C} | 1 | 0.409 | 0.332 |
| Vitamin D3 | k_{D3} | 0.409 | 1 | 0.150 |
| Vitamin A | k_{A} | 0.332 | 0.150 | 1 |
| ASLT | | | | |
| Vitamin C | k_{C} | 1 | 1.000 | 0.999 |
| Vitamin D3 | k_{D3} | 1.000 | 1 | 0.999 |
| Vitamin A | k_{A} | 0.999 | 0.999 | 1 |
| Control | | | | |
| Vitamin C | k_{C} | 1 | 0.991 | 0.993 |
| Vitamin D3 | k_{D3} | 0.991 | 1 | 0.986 |
| Vitamin A | k_{A} | 0.993 | 0.986 | 1 |

Table 8 shows a correlation coefficient matrix of parameters k_{C}, k_{D3} and k_{A}. Mathematical modeling based on the diffusion-reaction mechanism with oxygen and degradation of nutrients has been reported in the literature for various packaging materials, including high-density polyethylene (HPDE), Tetra Brik Aseptic cartons, and PET.

The parameter estimation of rate constants using the mechanistic model for vitamin degradation in samples kept at ALST and control resulted in a wide confidence interval (Table 7) and a high correlation between parameters (Table 8). This indicates that the mechanistic model is not suitable to represent the vitamin degradation in ASLT and control samples. The amount of oxygen diffusing into the samples in ASLT and control was at least 10 times and 15 times smaller compared to the amount of oxygen diffusing into samples kept at the UASLT condition. The mechanistic model represented in equations 20-23 was not sensitive to detecting oxygen amounts below a certain threshold which is reflected in the estimation of the rate of vitamin degradation in the ASLT and control samples. Though a minimal amount of oxygen was available for degradation reactions of vitamin C, D3 and A in the samples kept at ASLT and control, a substantial reduction in vitamins was observed as shown in FIGS. 7A through 7D. The degradation reactions were a result of reactions with any residual oxygen present initially and then followed anaerobic degradation in the absence of oxygen while reacting with the oxygen diffusing through the bottles, as explained above. Since the mechanistic model was not appropriate to model the degradation reactions in the ASLT and control, the 1^{st} order degradation model can be considered as reported in the literature for the degradation of vitamin A, vitamin C, and vitamin D3. The 1^{st} order model can be used to estimate the rate of reaction for the vitamin degradation in samples kept at the UASLT, ASLT, and control conditions. It can also be utilized for the development of predictive models for shelf-life extrapolation at room conditions based on the data obtained from the UASLT treatment.

Though more oxygen was diffusing under UASLT conditions (1.161×10⁻¹³ m²s⁻¹), the levels of headspace and dissolved oxygen did not go above 2% throughout the study. Since the headspace oxygen level did not increase over time, it indicates that oxygen solubilization occurred at the interface of the headspace and food as shown in FIG. 6. Since the UASLT samples did not accumulate oxygen levels beyond 21% (atmospheric level), this method can be considered to induce an appropriate aging process on food samples. Exposing food samples to oxygen levels beyond 21% oxygen is not only unrealistic but also has shown to nullify the impact of oxygen concentration on the degradation reactions as the rate becomes a plateau when the oxygen level approaches 21%.

From these tests, it can be concluded that according to aspects of the present invention exemplified by the UASLT method described herein, the application of oxygen pressure enhanced the diffusion of oxygen to the internal environment of the PET bottles. In the UASLT method, the food sample experienced an appropriate aging process because the oxygen level inside the packaging was maintained below 21% (atmospheric level) and only moderate temperature (40° C.) was used. The rapid influx of oxygen in the UASLT treatment increased the rate of reactions in the food products and resulted in faster degradation of color and vitamins A, C, and D3. The rapid degradation of vitamins and color in the samples from UASLT treatment was explained by the mechanistic model that coupled diffusion of oxygen and degradation of nutrients. In accordance with aspects of the invention as exemplified in the UASLT method, the elevated external oxygen pressure can be used as an accelerant along with moderate temperatures for rapid shelf-life testing of products in polymeric packaging. This new multi-accelerant approach has potential application in the food industry for faster shelf-life analysis of food and package selection.

A predictive model according to aspects of the present invention for the extrapolation of shelf-life at room conditions is provided that can be used with the UASLT method described herein. The predictive model can be used to predict (estimate) the shelf life of food at room conditions (estimated shelf-life). Since the UASLT method is based on temperature and oxygen diffusion, a model that correlates the diffusion of oxygen at a given temperature and the corresponding rate of vitamin degradation can be used for the shelf-life prediction model. A predictive model based on modification of the Arrhenius law, by introducing parameter(s) for temperature- and pressure-dependent diffusion, may be used for shelf-life prediction since the temperature-dependent oxygen consumption by nutrients is reported to follow the Arrhenius behavior. The predictive model that can be used with the UASLT method may be a temperature and oxygen diffusion-based model for shelf-life prediction of a model food at room conditions.

Two temperature-oxygen diffusion models were developed for shelf-life prediction of model food to be used with the UASLT method, a modified Arrhenius model and a modified Van't Hoff Arrhenius model. The estimated reaction rate constant and the predicted shelf-lives for vitamin degradations from the temperature-oxygen diffusion model matched closely with the conventional temperature-based Q₁₀ and Arrhenius models. Both of these temperature-oxygen diffusion models have the potential to be used with the UASLT method for shelf-life prediction.

The modified Arrhenius and modified Van't Hoff-Arrhenius models are temperature-oxygen diffusion models, whereas the Q₁₀ model and the Arrhenius model were temperature models. Comparisons were made with the conventional temperature-based models of Q₁₀ and Arrhenius, and the modified Arrhenius model and the modified Van't Hoff Arrhenius model provided good predictive estimates of the actual shelf-life. Development of these models is described hereinafter.

Kinetic parameters were estimated. Degradation of vitamins A, C and D3, and changes in the L* value were modeled using the 1^{st} order (equation 28 where Cₜ is the response variable at time t, C₀ is the initial value of the response variables, k is the 1^{st} order reaction rate constant (day⁻¹). Degradation of vitamin B1 was omitted from kinetic analysis and shelf-life prediction only minimal degradation was seen (FIGS. 7A through 7D).

$$\frac{{C}_{t}}{{C}_{o}} = exp ( - kt ) \qquad ( Eq . 28 )$$

Parameter k in equation 28 was estimated using the inverse problems approach based on the experimental values of vitamins and the L* value. Equation 28 was used to obtain the predicted dependent variable for the estimation utilizing appropriate prior information of the parameters. The scaled sensitivity coefficients of the parameters, which indicate the changes in the response variables caused by perturbation, were evaluated. The sequential estimation of the parameters was developed using matrix inversion lemma based on the Gauss Minimization method. Maximum a posteriori (MAP) was used to derive the mathematical form of non-linear sequential estimation. The Gauss minimization method is shown in equation 29.

$$S = [ Y - Y ( β ) ] W [ Y - Y ( β ) ] + [ μ - β ] U [ μ - β ] \qquad ( Eq . 29 )$$

In equation 29, Y is the experimental response variable, Ý is the predicted response, μ is the prior information of parameter vector β, W is the inverse of the covariance matrix of errors, and U is the inverse covariance matrix of parameters. β were solved and reported along with its root mean square error (RMSE) and 95% confidence interval. The 95% confidence intervals of the parameter were calculated. Residuals were calculated by taking the difference between the experimental and predicted temperature at each time point.

Q₁₀, Arrhenius, modified Arrhenius, and modified Van't Hoff-Arrhenius models were considered for the shelf-life prediction. Q₁₀ and Arrhenius were temperature models, while the modified Arrhenius and modified Van't Hoff-Arrhenius models were temperature-oxygen diffusion models. The parameters k estimated from these models were applied in equation 30 for the shelf-life prediction of the model food.

$$ln ( \frac{{C}_{t}}{{C}_{o}} ) = - kt \qquad ( Eq . 30 )$$

The values of Q₁₀, and the k estimated from the remaining three models and the corresponding shelf-life per vitamin were reported.

In the Q₁₀ model, the shelf-life estimation based on the Q₁₀ was performed as shown in equation 31.

$${Q}_{10}^{Δ T / 10} = \frac{{k}_{{T}_{1}}}{{k}_{{T}_{2}}} = \frac{Shelf - Life at {T}_{1}}{Shelf - Life at {T}_{2}} , Δ T = \vert - {T}_{1} - {T}_{2} \vert \qquad ( Eq . 31 )$$

The values of k_{T}_{₁} and k_{T}_{₂} at 22.5° C. and 40° C., respectively, were obtained from the 1^{st} order kinetic model as shown in equation 28 and were used to determine the Q₁₀ factor. The Q₁₀ value was then used to predict the shelf-life of the model food using equation 30.

In the Arrhenius model, the temperature dependence of the nutrient degradation reaction can be represented using the Arrhenius law (equations 32 or 33), where k and kₒ is the rate constant (day⁻¹), E_{A} is the apparent activation energy (J·mol⁻¹).

$$k = {k}_{o} {exp}^{( - \frac{{E}_{A}}{RT} )} \qquad ( Eq . 32 )$$

$$ln ( k ) = ( - \frac{{E}_{A}}{R} ) ( \frac{1}{T} ) + ln ( {k}_{o} ) \qquad ( Eq . 33 )$$

The values of k_{T}_{₁} and k_{T}_{₂} at 22.5° C. and 40° C. were used to estimate the E_{A}. The k estimated at 22.5° C. is then used to predict the shelf-life of the model food using equation 30.

The temperature-oxygen diffusion models for estimation of k according to some aspects of the invention are the Modified Arrhenius model and the Modified Van't Hoff-Arrhenius model described hereinafter.

In the Modified Arrhenius model, since oxygen diffusivity is dependent on both the temperature and the boundary oxygen pressure, the Arrhenius equation is modified to correlate k (day-1) with diffusivity (m²s-1) at a given temperature and boundary oxygen conditions as shown in equation 34.

$$k = A {exp}^{( - \frac{E}{D} )} \qquad ( Eq . 34 )$$

Equation 35 shows the linearized form where the plot of ln(k) vs 1/D was used to estimate k from the slope, E.

$$ln ( k ) = ( - E ) ( \frac{1}{D} ) + ln ( A ) \qquad ( Eq . 35 )$$

In both equations 34 and 35, A is the pre-exponential factor (day⁻¹) and E is the slope factor (m²s⁻¹). The k estimated at 22.5° C. using this correlation, is then used to predict the shelf-life of the model food using equation 30.

In the Modified Van't Hoff-Arrhenius model, temperature dependence of diffusion can be described by Van't Hoff-Arrhenius equation (equation 36), where Dₒ (m²s⁻¹) and E_{D} (J·mol⁻¹) are constants for the particular gas and polymer, respectively.

$$D = {D}_{o} exp ( - \frac{{E}_{D}}{RT} ) \qquad ( Eq . 36 )$$

E_{D} refers to the activation energy of diffusion. Combining equations 32 and 36 yields equations 37 or 38.

$$k = {{k}_{o} ( \frac{D}{{D}_{o}} )}^{( \frac{{E}_{A}}{{E}_{D}} )} \qquad ( Eq . 37 )$$

$$ln ( k ) = ( \frac{{E}_{A}}{{E}_{D}} ) ln ( D ) + [ ln ( {k}_{o} ) - ( \frac{{E}_{A}}{{E}_{D}} ) ln ( {D}_{o} ) ] \qquad ( Eq . 38 )$$

Plotting ln (k) against ln (D) results in a linear relationship, where the slope represents the ratio of E_{A}/E_{D} and the intercept represents ln(kₒ)−(E_{A}/E_{D})Ln(Dₒ). k can be estimated based on oxygen diffusion at the same experimental temperature and boundary oxygen pressure.

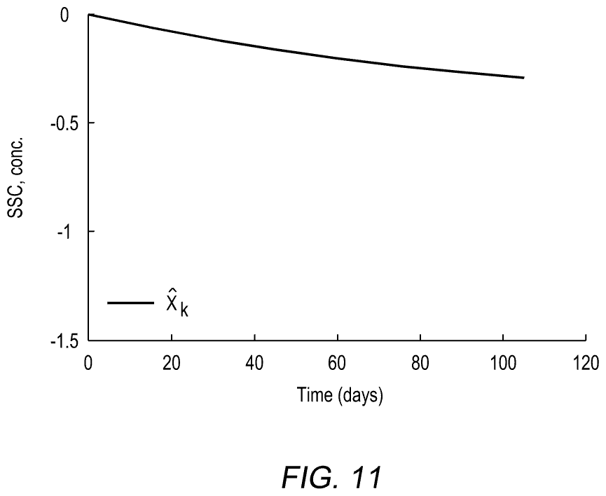

An example plot of the scaled sensitivity coefficient (SSC) of the parameter, k, estimated from the 1^{st} order reaction kinetic model, is shown in FIG. 11. The SSC indicates the sensitivity of the parameter(s) towards the changes in the response variables (i.e., nutrient concentration and L* value) which were analyzed prior to the experiments to ensure the parameters can be estimated properly.

The degradation of vitamins A, C, and D3 and the decrease in L* value in samples kept under UASLT, ASLT, and control conditions were subjected to the kinetic analysis using the 1^{st} order kinetic model. The k estimated from the 1^{st} order models were selected to evaluate the proposed shelf-life prediction models.

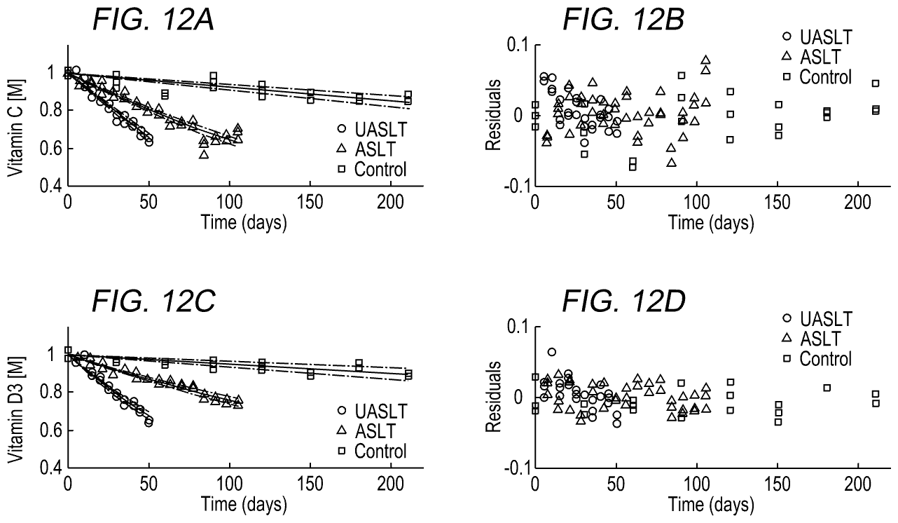

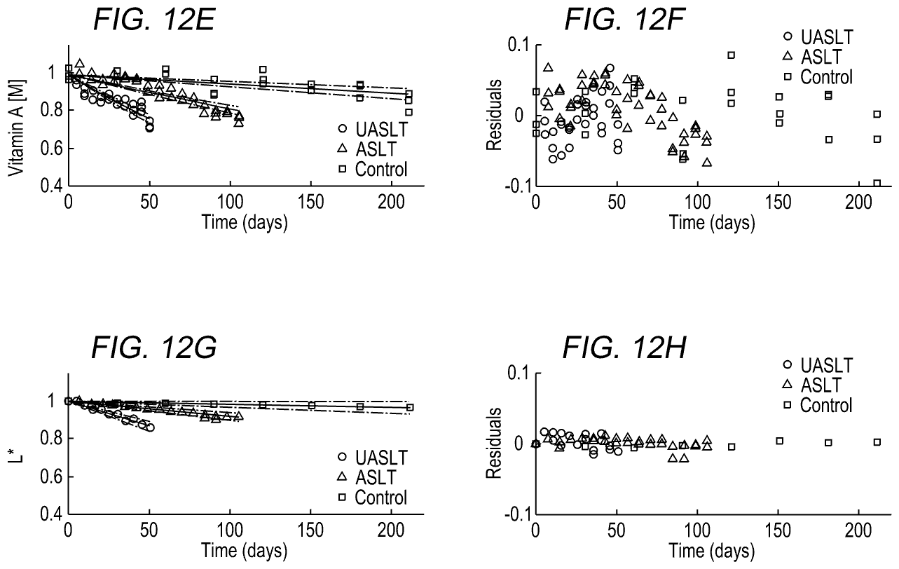

FIGS. 12A through 12H shows the degradation of vitamins A, C, and D3 and the decrease in L* value of samples fitted with the 1^{st} order kinetic model. Overall, the models seem to be appropriate to represent the degradation reactions in samples subjected to the UASLT, ASLT, and control conditions. The corresponding residual plots (FIGS. 12D, 12F, and 12H) show the errors to appear to have satisfied the standard statistical assumptions. An induction phase, a lag in degradation, was observed with degradation of vitamin A in samples kept under ASLT conditions, most likely due to the presence of vitamin C acting as an antioxidant by scavenging alkoxy radicals. The presence of vitamin C has been shown to slow down the degradation rate of vitamin A. The induction phase was not seen with samples subjected to the UASLT treatment since vitamin C was undergoing rapid degradation at this condition. The reaction rate constants, k, of the degradation of vitamins and the decrease in the L* values, are shown in Table 9.

TABLE 9

| | k × 10⁻² | Standard error | CI_{95%} × 10⁻² | RMSE |
|---|---|---|---|---|
| UASLT | | | | |
| Vitamin A | 0.55 | 0.09 | 0.50-0.59 | 0.03 |
| Vitamin C | 0.82 | 0.09 | 0.78-0.86 | 0.03 |
| Vitamin D3 | 0.77 | 0.09 | 0.73-0.81 | 0.03 |
| L* | 0.27 | 0.09 | 0.26-0.29 | 0.01 |
| ASLT | | | | |
| Vitamin A | 0.21 | 0.07 | 0.19-0.23 | 0.04 |
| Vitamin C | 0.44 | 0.08 | 0.42-0.46 | 0.03 |
| Vitamin D3 | 0.28 | 0.07 | 0.26-0.29 | 0.02 |
| L* | 0.08 | 0.07 | 0.08-0.09 | 0.01 |
| Control | | | | |
| Vitamin A | 0.05 | 0.05 | 0.04-0.07 | 0.04 |
| Vitamin C | 0.08 | 0.05 | 0.07-0.10 | 0.04 |
| Vitamin D3 | 0.05 | 0.05 | 0.04-0.06 | 0.02 |
| L* | 0.02 | 0.05 | 0.01-0.02 | 0.00 |

Table 9 shows estimation of k (day⁻¹) and the corresponding 95% confidence interval (CI_{95%}) and errors for degradation of vitamins A, C, and D3 with 1^{st} order reaction kinetics model, wherein CI_{95%}=95% confidence interval and RMSE=root means square error. The k value of L*, vitamin C, and vitamin D3 is doubled compared to the k value observed with the samples kept at the ASLT condition when oxygen pressure was applied as an accelerant. The k value for samples kept under ASLT which were 0.44, 0.28, and 0.08 day⁻¹ for vitamins C, D3, and L* increased to 0.82, 0.77, and 0.27 day⁻¹, respectively when oxygen pressure was introduced to the storage study along with a moderate temperature of 40° C. The k values reported for degradation of vitamins C and D3 in samples kept under ASLT conditions are in agreement with the literature. Since the UASLT methodology has not been reported before, no comparison with the literature is available.

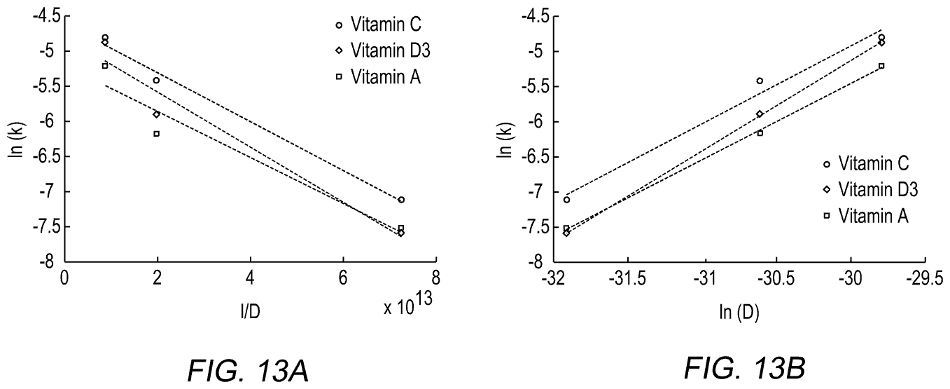

The shelf-life prediction of the model food was performed by calculating the duration (days) needed for vitamins C, D3 and A to undergo a 25% degradation at room temperature (22.5° C.). Two temperature-oxygen diffusion models were developed by correlating oxygen diffusion and rate of reaction based on the modified Arrhenius equation (Eq. 35) and the modified Van't Hoff-Arrhenius equation (Eq. 38). FIG. 13A shows the relationship between ln(k) and 1/D for vitamins C, D3 and A for the modified Arrhenius. The linear relationship shown is very similar to the relationship between the rate of reaction and the reciprocal temperature of the conventional Arrhenius equation. FIG. 13B shows a linear relationship between ln(k) and ln(D) in the modified Van't Hoff Arrhenius model for vitamins C, D3 and A. Each data point in both these plots represents the k and D estimated from experiments conducted at the same temperature and boundary oxygen pressure which included the data from UASLT, ASLT and control conditions. The slope and intercept of both plots were used to estimate k at 22.5° C. and 0.21 kPa oxygen based on the corresponding oxygen diffusivity, D. The k values were then used to estimate the duration for each vitamin to undergo 25% degradation using equation 30 as the shelf-life.

TABLE 10

| | Temperature-oxygen diffusion models | | | | | | | |
|---|---|---|---|---|---|---|---|---|
| | | | Modified Van't | | Temperature models | | | |
| | Modified Arrhenius | | Hoff Arrhenius | | Arrhenius model | | Q₁₀ model | |
| | k × 10⁻² | | k × 10⁻² | | k × 10⁻² | | Q₁₀ | |
| | (day⁻¹) | Days | (day⁻¹) | Days | (day⁻¹) | Days | factor | DaysHH |
| Vitamin C | 0.080 | 353 | 0.088 | 321 | 0.082 | 353 | 2.63 | 342 |
| Vitamin D3 | 0.048 | 583 | 0.051 | 548 | 0.051 | 563 | 2.62 | 529 |
| Vitamin A | 0.051 | 551 | 0.053 | 530 | 0.054 | 533 | 2.16 | 404 |

Table 10 shows predicted shelf-life (days) for 25% degradation of vitamins using temperature, and temperature-oxygen diffusion models. Both the estimated k and the shelf-life for each vitamin are presented in Table 10. For comparison, k values estimated from conventional temperature models, Q₁₀ and Arrhenius, are also presented in Table 10.

The k values estimated from the Arrhenius, modified Arrhenius and modified Van't Hoff-Arrhenius were close to each other for all vitamins. For vitamin C, the k values were in the range of 0.080-0.088 day⁻¹. These values are also significantly higher than the values estimated for other vitamins, as expected given vitamin C degradation was faster compared to other nutrients. The k values for vitamin D and A were in the range of 0.048-0.051 day⁻¹ and 0.051-0.054 day⁻¹, respectively. The Q₁₀ values estimated based on the rate of reactions at ASLT and control temperature for vitamins C, D3, and A are 2.63, 2.62 and 2.16, respectively. These values are consistent with values found in the literature for thermally processed foods. The predicted shelf-life of vitamin C to undergo 25% degradation was in the range of 321 to 353 days across all four models. The duration for vitamin D3 and A to fall to the same level was predicted to be in the range of 529 to 583 days and 404 to 551 days, respectively. The most conservative model for predicting the degradation of vitamin C was the modified Van′t Hoff-Arrhenius model. For both vitamins D3 and A, the Q₁₀ model appears to be the most conservative. Prediction of degradation of vitamins across all models is close to each other except for the shelf-life of vitamin A predicted with the Q₁₀ model. The duration for 25% degradation of vitamin A estimated from the Q₁₀ model 404 which is quite far from other models (530-551 days).

The shelf-life predicted with the temperature-diffusion models of the present invention appeared to be close to the conventional temperature model which indicates these models are appropriate to be used for shelf-life prediction. Both the modified Arrhenius and the modified Van't Hoff Arrhenius models disclosed herein appear not to have not been reported in the literature before and are a novel contribution of this study. The model was developed based on the evidence that the oxygen consumption by nutrients at various temperatures follows the Arrhenius behavior. These models have the potential to be used along with the UASLT method for rapid shelf-life determination and prediction of a food product.

The UASLT method disclosed herein is a promising method for inducing rapid degradation of vitamins as shown previously. While it has the potential for rapid shelf-life determination, an appropriate model is useful for shelf-life prediction of the product at any other product storage conditions. The UASLT method accelerated the degradation reaction by increasing the amount of oxygen diffusing into the packaging materials and food matrix from a high-oxygen-pressure environment. Since the samples kept at the UASLT condition had high oxygen diffusivity value and had resulted in a high rate of degradation reactions, the shelf-life of the sample at room temperature can be calculated by correlating the oxygen diffusivity values and rate of degradation reactions at the same experimental conditions. Since the UASLT experiments were conducted at a slightly elevated temperature, temperature differences are accounted for as well, besides the oxygen, when predicting shelf-life at room temperature. The appropriate temperature-oxygen diffusion model that has minimal errors can be selected with appropriate experimentation.

The new multi-accelerant approach of the present invention disclosed herein that combined the elevated temperature and high external oxygen pressure along with the temperature-oxygen diffusion predictive models may be used in the food industry to provide faster shelf-life analysis of food and package selection.

Methods within the scope of the invention are not necessarily limited to the specific materials, temperatures, pressures, equations, food products, or other details of the experiments described herein. Rather, methods described herein may be used at different temperatures (e.g., about 25 to about 40° C.) and/or different oxygen pressure levels, may be used with different packaging materials, for example, containing various thicknesses of EVOH layer, and may be used with other shelf-stable food products with different vitamin concentrations, pH, viscosity, antioxidants. Methods described herein may also be used with other predictive models.

In view of the foregoing, it can be seen that a multi-accelerant approach that combines oxygen pressure at an elevated temperature of 40-45° C. can increase the rate of the aging process via increased oxygen transfer. The use of a multiple-accelerant approach according to the present invention can be used as part of a rapid shelf-life predictor/method that can reduce the overall shelf-life analysis time compared to the conventional practice. An accompanying predictive model provides for shelf-life extrapolation at a given condition and time.

As previously noted above, though the foregoing detailed description describes certain aspects of one or more particular embodiments of the invention, alternatives could be adopted by one skilled in the art As such, and again as was previously noted, it should be understood that the invention is not necessarily limited to any particular embodiment described herein or illustrated in the drawings.

1. A method of estimating shelf-life of a packaged food product in a sealed polymeric package, the method comprising:
maintaining the sealed polymeric package in an oxygen atmosphere at a pressure greater than surrounding ambient atmospheric pressure during a test period of time;
maintaining the sealed polymeric package at a temperature above room temperature during the test period;
measuring a level of degradation of the packaged food product inside the sealed polymeric package at the end of the test period; and
extrapolating an estimated shelf-life of the packaged food product as a function of the amount of degradation measured, the temperature, and the pressure.

2. The method of claim 1, wherein the pressure is at a known constant pressure.

3. The method of claim 2, wherein the known constant pressure is approximately 20 psig.

4. The method of claim 1, wherein the temperature is a known constant temperature

5. The method of claim 4, wherein the known constant temperature is less than or equal to about 45° C.

6. The method of claim 1, wherein the test period is a preselected period of time.

7. The method of claim 1, wherein the test period varies and is dependent on the packaged food product reaching a preselected level of degradation.

8. The method of claim 1, wherein the degradation comprises a change of color of the packaged food product.

9. The method of claim 1, wherein the degradation comprises change of a nutrient of the packaged food product.

10. The method of claim 10, wherein the nutrient comprises a vitamin.

11. The method of claim 1, wherein the step of extrapolating comprises applying a temperature-oxygen diffusion model.

12. The method of claim 11, wherein the temperature-oxygen diffusion model is a modified Arrhenius model.

13. The method of claim 11, wherein the temperature-oxygen diffusion model is a modified Van't Hoff Arrhenius model.

14. The method of claim 1, wherein the food product comprises at least one of a food or a beverage.
